SEQUENCE LISTING (14 sequences)

SEQ ID NO: 1 - protein, 461 aa, Mycobacterium brisbanense JK1
MSTIAITNVTLIDGLGGLPRPATTVIVEGDRFATVGPSDSTPVPEGATVVDGNRRWMVPGYVNGNVHLLDAWMFMAGPGTIEYLARWEGRYVEVIEEAAQLALRNGVTTVFDTHNAIEPVLAARDRINAGISQGARIFAAGTIVGMGGPFSADFHFAGRTAATRTFVDRIDSMFEAGVGHQLSLLPRKEVRARVRDYLSRGVDMLKIAVSDHIVFTLVDRSVGFDRSYQTFSRPVLEVMVEEARAAGVPVLTHSVSVEALDTSVELGADVLIHANYTLGQEIPNYLIDKIVASDSWAGLQTVHDQHRQGLEDVGSWAAALAGEPYATNERNLISANAKILLNTDAGCPSKDHLADLSPVEREDRPWTIGNDHFHWTQSMVEKGMSPLEAISAATINVARAYGKADQIGSVETGKLADFVLLDQDPVDDIRNLRSITEVFQAGAAVDRAALPTTPLVTAHPA

SEQ ID NO: 2 - DNA, 1386 bp, Mycobacterium brisbanense JK1
atgagcaccatcgccatcaccaatgtcacgttgatcgacgggttgggcggcctgcccaggcccgccaccaccgtcatcgtcgagggagatcggttcgcgaccgtcggcccgtccgacagcacaccagtgccggagggcgccaccgtcgttgacggcaaccgccggtggatggtgcccggttacgtcaacggaaacgtccacctgctcgacgcatggatgttcatggccgggccgggcaccatcgaatacctggcccgatgggaaggacggtatgtcgaggtcatcgaggaggccgcgcagctggctctccgcaatggcgtaaccacagtgttcgacacgcacaacgcgatcgaacccgtactcgcagcgcgtgaccggatcaacgcgggcatctcacaaggcgcacgaatcttcgccgccggcaccatcgtcgggatgggcggaccgttcagcgcagacttccacttcgccggacgaacagcagccacgcggacgttcgtagaccgtatcgactcgatgttcgaggccggggtcggccaccagctcagtctgctgccacgcaaggaggtccgcgcccgcgtgcgcgattacctcagccgcggcgtagacatgctcaagatcgcggtcagcgaccacatcgtgttcacgctcgtcgaccgctcggtcggattcgaccgctcgtaccagacgttcagccgtcccgtcctcgaggtcatggtcgaggaagctcgggcggcaggagtaccggttctgacgcacagcgtgtccgttgaagctctcgacacctcggtggaactcggagccgacgtgctgatccatgcgaattacactcttggacaggagatcccgaactacttgatcgacaaaatcgtcgccagtgactcgtgggcggggctgcagaccgtgcatgaccagcaccgtcagggtctcgaagatgtcggaagctgggcggccgcgctcgccggcgagccgtatgcaaccaatgagcgcaatctgatcagtgccaatgccaagatcctgctgaacaccgatgctggttgtccgagcaaggaccatctcgccgacctgtccccggtcgagcgcgaggaccgtccctggacgatcggtaacgaccactttcactggactcagtcgatggtcgagaaaggcatgtccccccttgaagccatctccgcggccaccatcaacgtcgctcgcgcctacggcaaggccgaccagatcggctcggtcgagactgggaagctcgccgacttcgttctcctcgatcaagacccggtcgacgatatccgcaatctgcgatcaatcaccgaggtcttccaagccggcgcggccgtcgaccgggcagcgctgccgacgactccgctcgtgactgctcaccccgcgtaa

SEQ ID NO: 3 - protein, 456 aa, Arthrobacter globiformis D47
MTTTAITDITLIDGRGGAPERGVTVLVDEDRFSAIGPTATTPIPDGARVVGGAGRWMVPGYVNGNVHLLDAWMFMVGPGTIEYLARWEGRYVEVIEEAAQLVLRNGVTTVFDTYNAIEPVLAARDRINAGTSAGARIFAAGTIVGLGGPFSADFHFIGQQAASRTFVNRIDAMFEAGVGHQLSLLPRNEVRSRVRDYLARGVDMLKIAVSDHIVFTVGFDRSCQTFSRPVLEVMFEEARAAGVPVLTHSVSVEALDTSVDLGADVLIHANYTLGQHIPDYLIDKIAASDSYAGLQTVHHEHREGLERVGSWAATLAGEPYASNERALIRAKAKILMNTDAGCPSKDHLADLSPAEREDRPWTIGGDHFHWTQSIVEKGMTPLEAISAATLNVARAYGKDADIGSVETGKLADFVLLDADPTADIRHLRSISAVYQAGVAVDRGALPTTPLVTAHPA

SEQ ID NO: 4 - DNA, 1371 bp, Arthrobacter globiformis D47
atgaccaccaccgccatcaccgacatcaccctgatcgatggacgaggaggggcgcccgaacgcggagtgaccgttctcgtcgacgaggaccgcttctcggccatcggcccgaccgccaccacgcccatccccgacggcgcccgcgtcgtcggcggcgccggtcggtggatggtgccgggctacgtcaacggcaatgtgcacctcctcgacgcatggatgttcatggtcgggcccggcacgatcgagtacctcgcgcggtgggaggggcgctacgtcgaggtgatcgaggaagccgcgcagctcgtgctgcgcaacggcgtgacgaccgtgttcgacacgtacaacgcgatcgagcccgtgctggccgcgcgcgaccgcatcaacgcgggcacctccgccggcgcccgcatcttcgccgcgggaaccatcgtcggcctgggcggaccgttcagcgccgatttccacttcatcgggcagcaggcggcgtcgcggacgttcgtcaaccgcatcgacgccatgttcgaggccggggtggggcatcaactgagcctcctgccccgcaacgaggtgcgttcgcgcgtgcgggactatctcgctcgcggcgtcgacatgctgaagatcgccgtcagcgaccacatcgtcttcaccgtcgggttcgaccgctcgtgccagaccttcagccgccccgtgctcgaggtcatgttcgaggaagcgcgagctgcgggggtgccggtgctgacgcacagcgtgtcggtggaggcgctcgacacctcggtcgacctcggtgccgacgtcctgatccacgcgaactacacgctcggacagcacatcccggactacctcatcgacaagatcgccgcgagcgactcttacgcggggctgcagaccgtgcaccacgagcaccgtgaggggctcgagcgggtcggaagctgggcggcgacgctcgcgggggagccgtacgcgtcgaacgagcgcgcgttgatccgtgccaaggcgaagatcctcatgaacaccgatgccggctgcccgagcaaggatcacctcgccgacctgtcgcccgcggagcgcgaggatcgaccctggacgatcggcggcgaccacttccactggacccagtccatcgtggagaagggcatgaccccgctggaggcgatctcggcggcgacgctgaacgtcgcgcgcgcctacggcaaggatgccgacatcggctcggtcgagacgggcaagctcgcggacttcgtgctgctcgacgccgaccccaccgccgacatccgtcacctgcgctccatcagcgccgtgtaccaggccggtgtcgccgtcgaccgtggtgccctgcccaccacgccgctcgtcacggcgcatcccgcctga

SEQ ID NO: 5 - DNA, 33 bp, Artificial Sequence
  oligonucleotide/primer
ggcccatatgaccaccaccgccatcaccgacat

SEQ ID NO: 6 - DNA, 34 bp, Artificial Sequence
  oligonucleotide/primer
tgacggatcctcaggcgggatgcgccgtgacgag

SEQ ID NO: 7 - DNA, 32 bp, Artificial Sequence
  oligonucleotide/primer
ggcccatatgagcaccatcgccatcaccaatg

SEQ ID NO: 8 - DNA, 34 bp, Artificial Sequence
  oligonucleotide/primer
tgacggatccttacgcggggtgagcagtcacgag

SEQ ID NO: 9 - DNA, 31 bp, Artificial Sequence
  oligonucleotide/primer
tgacggatccaatgggcagcagccatcatca

SEQ ID NO: 10 - DNA, 35 bp, Artificial Sequence
  oligonucleotide/primer
ggccggatccaatgaccaccaccgccatcaccgac

SEQ ID NO: 11 - DNA, 36 bp, Artificial Sequence
  oligonucleotide/primer
ggccggatccaatgagcaccatcgccatcaccaatg

SEQ ID NO: 12 - DNA, 34 bp, Artificial Sequence
  oligonucleotide/primer
tgacaagctttcaggcgggatgcgccgtgacgag

SEQ ID NO: 13 - DNA, 34 bp, Artificial Sequence
  oligonucleotide/primer
tgacaagcttttacgcggggtgagcagtcacgag

SEQ ID NO: 14 - protein, 412 aa, Alteromonas macleodii
MSLDVDSKTLIHAGKLIDGKSDQVQSRISIVIDGNIISDIKKGFISSNDFEDYIDLRDHTVLPGLMDMHVHFGQEYQSKAQAPIKVEREMQAILATQHAYVTFKSGFTTVRQVGDSGLVAISLRDAINSGKLAGPRIFAAGKTIATTGGHADPTNGKAVDDYDYPVPEQGVVNGPYEVYAAVRQRYKDGADGIKITVTGGVLSVAKSGQNPQFTQEEVDAVVSAAKDYGMWVAVHAHGAEGMKRAIKAGVDSIEHGTFMDLEAMDLMIENGTYYVPTISAGEFVAEKSKIDNFFPEIVRPKAASVGPQISDTFRKAYEKGVKIAFGTDAGVQKHGTNWKEFVYMVENGMPAMKAIQSATMETAKLLRIEDKLGSIESGKLADLIAVKGNPIEDISVLENVDVVIKDGLLYEG

(19) United States
(12) Patent Application Publication
(10) Pub. No.: US 2011/0314564 A1
(43) Pub. Date: Dec. 22, 2011

(54) ENZYMES AND METHODS FOR HYDROLYSING PHENYLUREAS, CARBAMATES AND ORGANOPHOSPHATES

(75) Inventors: Jeevan L. Khurana, New South Wales (AU); Colin J. Jackson, Australian Capital Territory (NZ); Colin Scott, Australian Capital Territory (AU); Gunjan Pandey, Australin Capital Territory (AU); Robyn J. Russell, Australian Capital Territory (AU); John G. Oakeshott, Australin Capital Territory (AU)

(73) Assignee: Commonwealth Scientific and Industrial Research Organisation, Campbell, ACT (AU)

(21) Appl. No.: 13/002,846

(22) PCT Filed: Jul. 8, 2009
(86) PCT No.: PCT/AU2009/000883

§ 371 Date: May 27, 2011

Related U.S. Application Data
(60) Provisional application No. 61/134,442, filed on Jul. 9, 2008.

(51) Int. Cl. A62D 3/02; C12N 15/55; C12N 1/21; A01H 5/00; C12N 5/10; C12N 9/96; C12N 1/19; A01K 67/00; C12N 9/14; C12N 1/15

(52) U.S. Cl. 800/13; 435/195; 536/23.2; 435/320.1; 435/252.3; 435/254.11; 435/325; 435/419; 435/262.5; 435/188; 435/254.2; 435/252.33; 435/252.31; 435/254.21; 435/352; 435/350; 435/351; 435/364; 435/365; 435/354; 800/298

(57) ABSTRACT

The present invention relates to enzymes which are able to hydrolyse phenylureas, carbamates, and/or organophosphates, as well as polynucleotides encoding these enzymes. The present invention also relates to methods of hydrolysing phenylureas, carbamates, and/or organophosphates.

### FIELD OF THE INVENTION

The present invention relates to enzymes which are able to hydrolyse phenylureas, carbamates, and/or organophosphates, as well as polynucleotides encoding these enzymes. The present invention also relates to methods for hydrolysing phenylureas, carbamates, and/or organophosphates.

### BACKGROUND OF THE INVENTION

The phenylurea herbicide diuron (N′-3,4-dichlorophenyl N-dimethylurea) is a systemic photosynthesis inhibitor with a broad target range that is widely used in both cropped and non-cropped areas. The mode of action is via inhibition of the Hill reaction in photosynthesis (Wessels and Van der Veen, 1956), preventing oxygen production by binding at the reaction centre of photosystem II and blocking electron transfer (Stein et al., 1984; and Sinning, 1992).

Diuron is an environmental concern both because of off-site herbicidal effects and because of its toxicity, and that of its primary metabolite 3,4-dichloroaniline (DCA). Diuron and DCA are suspected to be genotoxic (Osano et al., 2002; and Canna-Michaelidou et al., 1996). Normal spraying practices (Gooddy et al., 2002) can result in leaching of diuron into ground (Spliid and Koppen, 1998; and Field et al., 2003), surface (Thurman et al., 2000; and Gerecke et al., 2001a), and ultimately, sea waters (Gerecke et al., 2001b). Because of the slow rate of chemical hydrolysis and photolysis of diuron (Salvestrini et al., 2002; and Okamura, 2002), it is environmentally persistent. Diuron was listed as a priority hazardous substance under review by Decision No 2455/2001/EC pursuant to the European Commission's Water Framework Directive (2000/60/EC) that recommended that diruron use be phased out by 2020. Subsequently, the European Commissions directive proposal (COM(2006)397) clarified that diuron is a priority substance of major concern in European waters for which emissions, discharges, and losses should be reduced.

Microbial degradation is thought to be a major route for the degradation of diuron, and the half-life in soil is estimated to be less than one year (Okamura, 2002; and Wauchope et al., 1992). In addition to microbial consortia and fungal isolates, a number of bacterial strains have been shown to catabolise a range of phenylurea herbicides, including diuron (Sorensen et al., 2003; and Giacomazzi and Cochet, 2004). For instance, Arthrobacter globiformis D47 degrades several phenylureas, in the order linuron>diuron>monolinuron>metoxuron>isoproturon, by rate (Cullington and Walker, 1999; Turnbull et al., 2001a). Subsequently, an Arthrobacter sp. N2 strain has been found to degrade diuron, chlorotoluron and isoproturon (Widehem et al., 2002; and Tixier et al., 2002). Neither of the Arthrobacter species entirely mineralise diuorn, and accumulate DCA as a result of diuron hydrolysis. In contrast, both Pseudomonas sp. strain Bk8 and Variovorax sp. SRS16 completely mineralise diuron (El-Deeb et al., 2000; Sorensen et al., 2008).

The first purified phenylurea hydrolase was found to be a 75 kDa enzyme from Bacillus sphaericus, which was reported to catalyse the breakdown of a number of N-methoxy-N-methyl phenylureas, including linuron, monolinuron, metobromuron, and chlorbromuron (Engelhardt et al., 1971; Engelhardt et al., 1973; and Wallnöfer, 1969). However, no turnover of N-dimethyl phenylurea substrates, including diuron, was observed. The only genetic characterisation of a hydrolytic diuron degrading gene to date has been that of Turnbull et al. (2001b), in which they cloned and sequenced a phenylurea hydrolase called puhA from A. globiformis D47. Sequence analysis showed that PuhA has low level similarity to members of the amidohydrolase superfamily (Turnbull et al., 2001b).

Enzymes in the amidohydrolase superfamily contain mono or binuclear metal centres within (β/α)₈-barrel structural fold, and catalyse the hydrolysis of amide or ester functional groups at carbon or phosphorus centres. The metal centres are located at the C-terminal end of the eight β-strands that constitute the barrel, with the protruding loops influencing substrate specificity. There are several subtypes of amidohydrolases, which are defined by the variations in the amino acids which function as direct metal ligands (see Seibert and Raushel, 2005 for review).

There is a need for further enzymes which can be used to hydrolyse phenylureas such as diuron.

### SUMMARY OF THE INVENTION

The present inventors have identified a bacterial strain which is capable of hydrolysing phenylureas, carbamates, and/or organophosphates. Furthermore, the inventors have identified enzymes which can be used to hydrolyse phenylureas, carbamates, and/or organophosphates.

Accordingly, the present invention provides a substantially purified and/or recombinant polypeptide comprising:

i) an amino acid sequence as provided in SEQ ID NO:1,

ii) an amino acid sequence which is at least 83% identical to i), and/or

iii) a biologically active fragment of i) or ii),

wherein the polypeptide is capable of hydrolysing a phenylurea, carbamate, and/or organophosphate.

In an embodiment of the invention, the phenylurea is a N-dimethyl or N-methoxy-N-methyl substituted phenylurea. The N-dimethyl substituted phenylurea may be, for example, diuron, chlortoluron, fluomethuron, metoxuron, isoproturon, or fenuron. The N-methoxy-N-methyl substituted phenylurea may be, for example, linuron, chlorbromuron, metobromuron, or monolinuron.

In a further embodiment of the invention, the polypeptide has a lower Kₘ for diuron, chlortoluron, fluomethuron, metoxuron, and/or fenuron than a polypeptide comprising an amino acid sequence as provided in SEQ ID NO:3.

In an embodiment of the invention, the polypeptide has at least a 2-fold, more preferably at least a 4-fold, lower Kₘ for diuron than a polypeptide comprising an amino acid sequence as provided in SEQ ID NO:3.

In an embodiment of the invention, the polypeptide has at least a 4-fold, more preferably at least an 8-fold, more preferably at least a 10-fold, lower Kₘ for chlortoluron than a polypeptide comprising an amino acid sequence as provided in SEQ ID NO:3.

In an embodiment of the invention, the polypeptide has at least a 2-fold, more preferably at least a 5-fold, lower Kₘ for fluomethuron than a polypeptide comprising an amino acid sequence as provided in SEQ ID NO:3.

In an embodiment of the invention, the polypeptide has at least a 2-fold, more preferably at least a 3-fold, lower Kₘ for metoxuron than a polypeptide comprising an amino acid sequence as provided in SEQ ID NO:3.

In an embodiment of the invention, the polypeptide has at least a 8-fold, more preferably at least a 12-fold, more preferably at least 16-fold, lower Kₘ for fenuron than a polypeptide comprising an amino acid sequence as provided in SEQ ID NO:3.

In an embodiment, the polypeptide hydrolyses an amide bond of the phenylurea, carbamate, and/or organophosphate.

In a preferred embodiment of the invention, the polypeptide comprises an amino acid sequence which is at least 95% identical to SEQ ID NO:1.

In a further preferred embodiment of the invention, the polypeptide can be purified from a Mycobacterium sp. Preferably, the Mycobacterium sp. is Mycobacterium brisbanense JK1 deposited under accession number V08/013277 on 16 May 2008 at the National Measurement Institute, Australia.

In an embodiment of the invention, the polypeptide is fused to at least one other polypeptide. The at least one other polypeptide may be, for example, a polypeptide that enhances the stability of a polypeptide of the present invention, or a polypeptide that assists in the purification of the fusion protein.

In a further aspect, the present invention provides an isolated and/or exogenous polynucleotide comprising:

i) a sequence of nucleotides as provided in SEQ ID NO:2,

ii) a sequence of nucleotides encoding a polypeptide of the invention,

iii) a sequence of nucleotides which is at least 80% identical to i),

iv) a sequence of nucleotides which hybridizes to i) under stringent conditions, and/or

v) a sequence of nucleotides complementary to any one of i) to iv).

Preferably, the polynucleotide encodes a polypeptide that hydrolyses a phenylurea, carbamate, and/or organophosphate.

In a further aspect, the present invention provides a vector comprising a polynucleotide of the invention.

Preferably, the polynucleotide is operably linked to a promoter.

In yet a further aspect, the present invention provides a host cell comprising at least one polynucleotide of the invention and/or at least one vector of the invention.

The host cell can be any type of cell. In one embodiment of the invention, the host cell is a plant cell. In another embodiment of the invention, the host cell is a bacterial cell.

Preferably, the polypeptide of the invention is produced by the cell by the expression of a polynucleotide of the invention.

In another aspect, the present invention provides a method for producing a polypeptide of the invention, the method comprising cultivating a host cell of the invention encoding said polypeptide, or a vector of the invention encoding said polypeptide, under conditions which allow expression of the polynucleotide encoding the polypeptide, and recovering the expressed polypeptide.

Also provided is a polypeptide produced using a method of the invention.

In a further aspect, the present invention provides an isolated antibody which specifically binds to a polypeptide of the invention.

In yet another aspect, the present invention provides a composition comprising at least one polypeptide of the invention, at least one polynucleotide of the invention, a vector of the invention, a host cell of the invention, and/or an antibody of the invention.

In a further aspect, the present invention provides a composition for hydrolysing a phenylurea, carbamate, and/or organophosphate, the composition comprising a polypeptide of the invention and/or a host cell of the invention.

Preferably, a composition of the invention further comprises one or more acceptable carriers.

Preferably, a composition of the invention further comprises metal ions. In a preferred embodiment of the invention, the metal ions are divalent metal ions. More preferably, the metal ions are selected from Mg²⁺, Co²⁺, Ca²⁺, Zn²⁺, Mn²⁺, and combinations thereof. More preferably, the metal ions are selected from Mg²⁺, Zn²⁺, Co²⁺, and combinations thereof. In a particularly preferred embodiment of the invention, the metal ions are Zn²⁺.

The polypeptides of the invention can be used as a selectable marker to detect a host cell. Thus, also provided is the use of a polypeptide of the invention, or a polynucleotide encoding said polypeptide, as a selectable marker for detecting and/or selecting a host cell.

In another aspect, the present invention provides a method for detecting a host cell, the method comprising

i) contacting a cell or a population of cells with a polynucleotide encoding a polypeptide of the invention under conditions which allow uptake of the polynucleotide by the cell(s), and

ii) selecting a host cell by exposing the cells from step i), or progeny cells thereof, to a phenylurea, carbamate, and/or organophosphate.

Preferably, the polynucleotide comprises a first open reading frame encoding a polypeptide of the invention, and a second open reading frame not encoding a polypeptide of the invention.

In one embodiment, the second open reading frame encodes a polypeptide. In a second embodiment, the second open reading frame encodes a polynucleotide which is not translated. In both instances, it is preferred that the second open reading frame is operably linked to a suitable promoter.

The polynucleotide which is not translated may encode, for example, a catalytic nucleic acid, a dsRNA molecule, or an antisense molecule.

Examples of a suitable cell include, but are not limited to, a plant cell, bacterial cell, fungal cell, or animal cell. Preferably, the cell is a plant cell.

In a further aspect, the present invention provides a method for hydrolysing a phenylurea, carbamate, and/or organophosphate, the method comprising contacting a phenylurea, carbamate, and/or organophosphate with a polypeptide of the invention.

In one embodiment of the invention, the polypeptide is produced by a host cell of the invention.

Polypeptides provided herein can be produced in plants to enhance the host plant's ability to grow when exposed to a phenylurea, carbamate, and/or organophosphate.

Thus, in yet a further aspect, the present invention provides a transgenic plant comprising an exogenous polynucleotide encoding at least one polypeptide of the invention.

Preferably, the polynucleotide is stably incorporated into the genome of the plant.

In a further aspect, the present invention provides a part of a transgenic plant of the invention. Preferably, the part of the transgenic plant is seed.

In another aspect, the present invention provides a method for hydrolysing a phenylurea, carbamate, and/or organophosphate in a sample, the method comprising contacting the sample with a transgenic plant of the invention.

Preferably, the sample is soil. Such soil can be in a field.

In another aspect, the present invention provides a transgenic non-human animal comprising an exogenous polynucleotide encoding at least one polypeptide of the invention.

In a further aspect, the present invention provides an isolated strain of Mycobacterium deposited under accession number V08/013277 on 16 May 2008 at the National Measurement Institute, Australia.

The strain may be alive or dead (killed).

In yet another aspect, the present invention provides a composition for hydrolysing a phenylurea, carbamate, and/or organophosphate, the composition comprising a strain of the invention and optionally, one or more acceptable carriers.

In a further aspect, the present invention provides an extract of a host cell of the invention, a transgenic plant of the invention, a transgenic non-human animal of the invention, or a strain of the invention, wherein the extract comprises a polypeptide of the invention.

In yet another aspect, the present invention provides a composition for hydrolysing a phenylurea, carbamate, and/or organophosphate, the composition comprising an extract of the invention, and optionally, one or more acceptable carriers.

In a further aspect, the present invention provides a method of hydrolysing a phenylurea, carbamate, and/or organophosphate, the method comprising contacting a phenylurea, carbamate, and/or organophosphate with a strain of the invention, a composition of the invention, and/or an extract of the invention.

In another aspect, the present invention provides a polymeric sponge or foam for hydrolysing a phenylurea, carbamate, and/or organophosphate, the foam or sponge comprising a polypeptide of the invention immobilized on a polymeric porous support.

Preferably, the porous support comprises polyurethane.

In a preferred embodiment, the sponge or foam further comprises carbon embedded or integrated on or in the porous support.

In a further aspect, the present invention provides a method for hydrolysing a phenylurea, carbamate, and/or organophosphate, the method comprising contacting a phenylurea, carbamate, and/or organophosphate with a sponge or foam of the invention.

The polypeptides of the present invention can be mutated, and the resulting mutants screened for altered activity, such as enhanced enzymatic activity. Such mutations can be performed using any technique known in the art including, but not limited to, in vitro mutagenesis and DNA shuffling.

Thus, in a further aspect, the present invention provides a method of producing a polypeptide with enhanced ability to hydrolyse a phenylurea, carbamate, and/or organophosphate, or altered substrate specificity for a different type of phenylurea, carbamate, and/or organophosphate, the method comprising:

i) altering one or more amino acids of a first polypeptide of the invention,

ii) determining the ability of the altered polypeptide obtained from step i) to hydrolyse a phenylurea, carbamate, and/or organophosphate, and

iii) selecting an altered polypeptide with enhanced ability to hydrolyse a phenylurea, carbamate, and/or organophosphate, or altered substrate specificity for a different type of phenylurea, carbamate, and/or organophosphate, when compared to the polypeptide used in step i).

Step i) can be performed using any suitable technique known in the art such as, but not limited to, site-directed mutagenesis, chemical mutagenesis and DNA shuffling on the encoding nucleic acid.

Also provided is a polypeptide produced by a method of the invention.

In yet another aspect, the present invention provides a method for screening for a microorganism capable of hydrolysing a phenylurea, carbamate, and/or organophosphate, the method comprising:

i) culturing a candidate microorganism in the presence of a phenylurea, carbamate, and/or organophosphate, as a sole nitrogen source, and

ii) determining whether the microorganism is capable of growth and/or division.

In a further aspect, the present invention provides a kit comprising at least one polypeptide of the invention, at least one polynucleotide of the invention, a vector of the invention, a host cell of the invention, an antibody of the invention, a composition of the invention, at least one part of a plant of the invention, at least one strain of the invention, at least one extract of the invention, and/or at least one polymeric sponge or foam of the invention.

In yet a further aspect, the present invention provides a method for hydrolysing a carbamate and/or an organophosphate, the method comprising contacting a carbamate and/or an organophosphate with a substantially purified and/or recombinant polypeptide comprising:

i) an amino acid sequence as provided in SEQ ID NO:1 or SEQ ID NO:3,

ii) an amino acid sequence which is at least 40% identical to i), and/or

iii) a biologically active fragment of i) or ii),

wherein the polypeptide hydrolyses a carbamate and/or an organophosphate.

It will be understood by the skilled person in the art that the method can be carried out by contacting a carbamate and/or an organophosphate with a polynucleotide encoding the polypeptide, a vector and/or host cell comprising a polynucleotide encoding the polypeptide, a host cell comprising the vector, a transgenic plant or part thereof comprising a polynucleotide encoding the polypeptide, a transgenic non-human animal comprising the polynucleotide encoding the polypeptide, an isolated strain of Mycobacterium deposited under accession number V08/013277 on 16 May 2008 at the National Institute Australia, an extract of the host cell, transgenic plant, transgenic non-human animal, or strain wherein the extract comprises the polypeptide, and/or a polymeric sponge or foam comprising the polypeptide.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, group of steps or group of compositions of matter.

Each embodiment described herein is to be applied mutatis mutandis to each and every other embodiment unless specifically stated otherwise.

The invention is hereinafter described by way of the following non-limiting Examples and with reference to the accompanying figures.

### BRIEF DESCRIPTION OF THE ACCOMPANYING DRAWINGS

FIG. 1. Hydrolysis of diuron and accumulation of DCA by Mycobacterium brisbanense strain JK1. Diuron concentrations in the 3 replicate flasks was plotted (Δ,□,∘) with the closed symbols (▴,▪,) representing DCA concentrations in the same 3 flasks. No loss of diuron was observed in media only controls (×).

FIG. 2. Synteny between puhA and puhB and a putative regulatory gene tetR. The puhA and puhB genes are represented by arrows indicating the direction of transcription relative to a conserved putative tetR family transcriptional regulator. Predicted promoter regions (−10, −35) and ribosome binding sites (RBS) upstream of the puhA and puhB genes are shown. A 14 bp palindromic sequence upstream of puhA (indicated with inverted arrows) is similar to an imperfect palindromic sequence upstream of puhB.

FIG. 3. A neighbour-joining phylogeny of PuhA and B amongst 32 of the most diverse members of the metal dependant hydrolases group A (CD01299). Numbers at nodes are bootstrap resampling frequencies from 1000 replicates.

FIG. 4. Sequence alignment of 2QS8 and PuhB for homology modelling. Metal ligands are shown in bold, second shell catalytic residues are shown in bold and underlined.

FIG. 5. Homology model of PuhB based on 2QS8. The PuhB model is shown (left) alongside the active site of 2GOK. The model is based on the alignment shown in FIG. 4.

FIG. 6. Purification of PuhA and PuhB expressed in M. smegmatis. A 10% SDS-PAGE gel loaded with samples containing roughly equivalent quantities of the target protein from each step of the purification; cell free extract (CFE), hydrophobic interaction chromatography (HIC), anion exchange chromatography (AEX), and size exclusion chromatography (SEC).

FIG. 7 Effects of reaction temperature on the activity of PuhA () and B (Δ). Assays were performed for 1 hour at the designated temperature. Error bars are standard deviations of triplicate assays.

FIG. 8. Stability of PuhA () and B (▴). Residual activities resulting from exposure to (A) various temperatures for 10 minutes and (B) the solvents acetonitrile [PuhA() and B(▴)] and methanol [PuhA(□) and B(Δ)] in a 1 hour assay at 25° C. Error bars are the standard deviations of triplicate assays.

FIG. 9. Kinetic data for the phenylurea hydrolases

FIG. 10. The pH dependences of PuhA (▪) and PuhB (▴). The pH dependence was measured with the substrate diuron in a constant ionic strength buffer and analysed by LCMS. Plots of pH vs. log (k_{cat}) (A), pH vs. log (k_{cat}/Kₘ) (B), and pH vs. Kₘ (C) demonstrate the broad pH optima of these enzymes.

FIG. 11. Proposed catalytic mechanism of the phenylurea hydrolases. H273 in the second shell of the active site coordinates the phenylurea substrate. Attack at the central carbon of the urea moiety of the substrate by a hydroxide activated by the active site metal occurs in concert with stabilization of the aniline leaving group by K206.

FIG. 12. Brønsted plots of PuhA (□) and PuhB (Δ). The k_{cat} and the leaving groups pKₐs of of the N-dimethyl phenylureas in FIG. 7 show a dependence.

### KEY TO SEQUENCE LISTING

SEQ ID NO:1—Amino acid sequence of PuhB
SEQ ID NO:2—Nucleotide sequence encoding PuhB
SEQ ID NO:3—Amino acid sequence of PuhA
SEQ ID NO:4—Nucleotide sequence encoding PuhA
SEQ ID NO:5—Primer (PuhA-pet14b fwd)
SEQ ID NO:6—Primer (PuhA-pet14b rev)
SEQ ID NO:7—Primer (PuhB-pET14b fwd)
SEQ ID NO:8—Primer (PuhB-pET14b rev)
SEQ ID NO:9—Primer (His AB pMV fwd)
SEQ ID NO:10—Primer (PuhA-pMV261 fwd)
SEQ ID NO:11—Primer (PuhB-pMV261 fwd)
SEQ ID NO:12—Primer (PuhA pMV261 rev)
SEQ ID NO:13—Primer (PuhB pMV261 rev)
SEQ ID NO:14—Amino acid sequence of 2QS8

### DETAILED DESCRIPTION OF THE PREFERRED EMBODIMENTS

### General Techniques

Unless specifically defined otherwise, all technical and scientific terms used herein shall be taken to have the same meaning as commonly understood by one of ordinary skill in the art (e.g., in cell culture, molecular genetics, plant biology and/or chemistry, recombinant cell biology including transgenic plants, bioremediation, immunology, immunohistochemistry, protein chemistry, and biochemistry).

Unless otherwise indicated, the recombinant protein, cell culture, and immunological techniques utilized in the present invention are standard procedures, well known to those skilled in the art. Such techniques are described and explained throughout the literature in sources such as, J. Perbal, A Practical Guide to Molecular Cloning, John Wiley and Sons (1984); J. Sambrook et al., Molecular Cloning: A Laboratory Manual, Cold Spring Harbour Laboratory Press (1989); T. A. Brown (editor), Essential Molecular Biology: A Practical Approach, Volumes 1 and 2, IRL Press (1991); D. M. Glover and B. D. Hames (editors), DNA Cloning: A Practical Approach, Volumes 1-4, IRL Press (1995 and 1996); F. M. Ausubel et al. (editors), Current Protocols in Molecular Biology, Greene Pub. Associates and Wiley-Interscience (1988, including all updates until present); Ed Harlow and David Lane (editors) Antibodies: A Laboratory Manual, Cold Spring Harbour Laboratory, (1988); and J. E. Coligan et al. (editors), Current Protocols in Immunology, John Wiley & Sons (including all updates until present).

As used herein, the term “hydrolase” refers to a polypeptide of the present invention that catalyses the hydrolysis of a phenylurea, carbamate, and/or organophosphate. As used herein, the term “hydrolysis” refers to a chemical process of decomposition involving the splitting of a bond and the addition of the hydrogen cation and hydroxide anion of water. To “hydrolyse” as used herein, is to subject to or undergo hydrolysis.

### Phenylureas

Phenylurea herbicides generally possess the general structure:

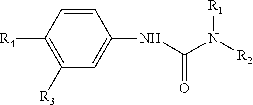

wherein, R₁ may be, for example, CH₃; R₂ may be, for example, CH₃ or OCH₃; R₃ may be, for example, H, Cl, or CF₃; and R₄ may be, for example, H, CH₃, OCH₃, CH(CH₃)₂, Cl, or Br.

The majority of the currently available phenylurea herbicides are either N-dimethyl-substituted (for example, diuron, chlorotoluron, flumethuron, metoxuron, isoproturon and fenuron) or N-methoxy-N-methyl- (for example, linuron, chlorobromuron, metobromuron and monolinuron) compounds. The phenylurea herbicides generally have relatively high solubility in water and low tendencies to sorb to soil, rendering them mobile in soil.

In a preferred embodiment, the polypeptides and methods of the present invention can be used to hydrolyse N-dimethyl ureas and/or N-methoxy-N-methyl ureas. In a particularly preferred embodiment of the invention, the polypeptides and methods of the present invention are used to hydrolyse diuron (N′-3,4-dichlorophenyl N-dimethylurea).

Typically, the polypeptides of the present invention convert phenylureas through the hydrolysis of the urea carbonyl group into aniline and carbamic acid, or in the case of substituted phenylureas, the corresponding substituted aniline (e.g., diuron is hydrolysed to 3,4-dichloroaniline (DCA) while isoproturon is hydrolysed to 4-isopropylaniline) and N-dimethyl or N-methoxy, N-methyl carbamic acid (which may be further degraded to CO₂ and dimethyl or methoxymethyl amine (Engelhardt, 1971)).

### Carbamates

Carbamates possess an amide linkage, with the carbonyl group also forming a carboxylester linkage. Carbamate pesticides are derived from carbamic acid (HOOC—NH₂) and possess the general structure:

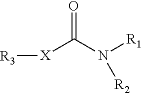

The chemical side chains principally govern the biological activity of the pesticide. The atom denoted by the X is either an O or a S, whereas R₁ and R₂ can be a number of different organic side chains, although quite often a CH₃ group or a H. R₃ is usually a bulky aromatic group or an oxime moiety.

Different constituents from either the amine group or the carboxyl ester group determine the target organism of these compounds. Carbamates with aromatic groups from both the amine and carboxylester (e.g., phenmedipham) are herbicidal. Carbamates with an aromatic group coming from the carboxylester group and a small group, such as a CH₃ group, coming from the amine (such as carbaryl) are insecticidal.

Carbamates with a benzimidazole group coming from the amine and a small CH₃ group coming from the carboxylester linkage are fungicidal. Such carbamates are referred to herein as benzimidazole carbamate fungicides and include, but are not limited to, benomyl, carbendazim, cypendazole, debacarb and mecarbinzid.

In a particularly preferred embodiment of the invention, the carbamates that can be hydrolysed by the methods of the present invention include linuron ester and DMNPC.

### Organophosphates

Organophosphates are synthetic organophosphorus esters and related compounds such as phosphoramidates. They have the general formula (RRX)P═O or (RR′X)P═S, where R and R′ are short-chain groups. For insecticidal organophosphates, X is a good leaving group, which is a requirement for the irreversible inhibition of acetylcholinesterase.

The polypeptides and methods of the present invention can hydrolyse the phosphotriester bonds of organophosphates.

Organophosphates that can be hydrolysed by the polypeptides and methods of the present invention include, for example, paraoxon ethyl.

Although well known for their use as pesticides, organophosphates have also been used as nerve gases against mammals. Accordingly, it is envisaged that the polypeptides and methods of the present invention will also be useful for hydrolysis of organophosphates which are not pesticides.

### Polypeptides

By “substantially purified polypeptide” or “purified polypeptide”, we mean a polypeptide that has been separated from one or more lipids, nucleic acids, other polypeptides, or other contaminating molecules with which it is associated in its native state. It is preferred that the substantially purified polypeptide is at least 60% free, more preferably at least 75% free, and more preferably at least 90% free from other components with which it is naturally associated.

The term “recombinant” in the context of a polypeptide refers to the polypeptide when produced by a cell, or in a cell-free expression system, in an altered amount or at an altered rate compared to its native state. In a preferred embodiment of the invention, the cell is a cell that does not naturally produce the polypeptide. In an alternate embodiment, the cell is a cell which comprises an exogenous polynucleotide that causes an altered, preferably increased, amount of the polypeptide to be produced. A recombinant polypeptide of the invention includes polypeptides which have not been separated from other components of the cell or cell-free expression system, in which it is produced, and polypeptides produced in such cells or cell-free systems which are subsequently purified away from at least some other components.

The terms “polypeptide” and “protein” are generally used interchangeably and refer to a single polypeptide chain which may or may not be modified by addition of non-amino acid groups. It would be understood that such polypeptide chains may associate with other polypeptides or proteins or other molecules such as co-factors. The terms “proteins” and “polypeptides” as used herein also include variants, mutants, modifications, analogues, biologically active fragments and/or derivatives of the polypeptides of the invention as described herein.

The % identity of a polypeptide is determined by GAP (Needleman and Wunsch, 1970) analysis (GCG program) with a gap creation penalty=5, and a gap extension penalty=0.3. The query sequence is at least 25 amino acids in length, and the GAP analysis aligns the two sequences over a region of at least 25 amino acids. More preferably, the query sequence is at least 50 amino acids in length, and the GAP analysis aligns the two sequences over a region of at least 50 amino acids. More preferably, the query sequence is at least 100 amino acids in length and the GAP analysis aligns the two sequences over a region of at least 100 amino acids. Even more preferably, the query sequence is at least 250 amino acids in length and the GAP analysis aligns the two sequences over a region of at least 250 amino acids. Even more preferably, the GAP analysis aligns the two sequences over their entire length.

As used herein, a “biologically active fragment” is a portion of a polypeptide of the invention which maintains a defined activity of the full-length polypeptide, namely be able hydrolyse a phenylurea, carbamate, and/or organophosphate. Biologically active fragments can be any size as long as they maintain the defined activity. Preferably, biologically active fragments are at least 100, more preferably at least 200, and even more preferably at least 350 amino acids in length.

With regard to a defined polypeptide, it will be appreciated that % identity figures higher than those provided above will encompass preferred embodiments. Thus, where applicable, in light of the minimum % identity figures, it is preferred that the polypeptide comprises an amino acid sequence which is at least 40%, more preferably at least 45%, more preferably at least 50%, more preferably at least 55%, more preferably at least 60%, more preferably at least 65%, more preferably at least 70%, more preferably at least 75%, more preferably at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO.

Amino acid sequence mutants of the polypeptides of the present invention can be prepared by introducing appropriate nucleotide changes into a nucleic acid of the present invention, or by in vitro synthesis of the desired polypeptide. Such mutants include, for example, a deletion, insertion or substitution of one or more residues within the amino acid sequence. A combination of one or more deletions, insertions and/or substitutions can be made to arrive at the final construct, provided that the final polypeptide product possesses the desired characteristics.

Mutant (altered) polypeptides can be prepared using any technique known in the art. For example, a polynucleotide of the invention can be subjected to in vitro mutagenesis. Such in vitro mutagenesis techniques include sub-cloning the polynucleotide into a suitable vector, transforming the vector into a “mutator” strain such as the E. coli XL-1 red (Stratagene) and propagating the transformed bacteria for a suitable number of generations. In another example, the polynucleotides of the invention are subjected to DNA shuffling techniques as broadly described by Harayama (1998). These DNA shuffling techniques may include genes related to those of the present invention, such as other hydrolases from bacteria, for example the genes encoding PuhA and PuhB could be subjected to shuffling. Products derived from mutated/altered DNA can readily be screened using techniques described herein to determine if they are able to confer the desired phenotype such as enhanced activity and/or altered substrated specificity.

In designing amino acid sequence mutants, the location of the mutation site and the nature of the mutation will depend on characteristic(s) to be modified. The sites for mutation can be modified individually or in series, for example, by (1) substituting first with conservative amino acid choices and then with more radical selections depending upon the results achieved, (2) deleting the target residue, or (3) inserting other residues adjacent to the located site.

Amino acid sequence deletions generally range from about 1 to 15 residues, more preferably about 1 to 10 residues and typically about 1 to 5 contiguous residues.

Substitution mutants have at least one amino acid residue in the polypeptide removed and a different residue inserted in its place. The sites of greatest interest for substitutional mutagenesis include sites identified as important for function. Other sites of interest are those in which particular residues obtained from various strains or species are identical. These positions may be important for biological activity. These sites, especially those falling within a sequence of at least three other identically conserved sites, are preferably substituted in a relatively conservative manner. Such conservative substitutions are shown in Table 1.

TABLE 1
Exemplary substitutions.

| Original | Exemplary |
|---|---|
| Residue | Substitutions |
| Ala (A) | val; leu; ile; gly |
| Arg (R) | lys |
| Asn (N) | gln; his |
| Asp (D) | glu |
| Cys (C) | ser |
| Gln (Q) | asn; his |
| Glu (E) | asp |
| Gly (G) | pro, ala |
| His (H) | asn; gln |
| Ile (I) | leu; val; ala |
| Leu (L) | ile; val; met; ala; phe |
| Lys (K) | arg |
| Met (M) | leu; phe |
| Phe (F) | leu; val; ala |
| Pro (P) | gly |
| Ser (S) | thr |
| Thr (T) | ser |
| Trp (W) | tyr |
| Tyr (Y) | trp; phe |
| Val (V) | ile; leu; met; phe; ala |

In a preferred embodiment, a polypeptide of the invention comprises a (β/α)₈ structural fold having a mononuclear metal centre. Preferably, the metal centre is within the catalytic site of the polypeptide (referred to herein as the “mononuclear active site”). Preferably, the mononuclear active site of the polypeptide comprises a Zn metal ion co-ordinated by an N×H motif from β-strand 1, an H from β-strand 5 and a D from β-strand 8. Preferably, the mononuclear active site also comprises an H on β-strand 6 which is located in the second shell of the active site. The mononuclear active site may also comprise a K residue that is not coordinated by the metal ion. In one embodiment, the polypeptide comprises an H at a position corresponding to amino acid number 253, a D at a position corresponding to amino acid number 334 and/or an H at a position corresponding to amino acid number 273 when compared to SEQ ID NO:1. In another example, the polypeptide additionally comprises a K at position corresponding to amino acid number 206 when compared to SEQ ID NO:1. Furthermore, when designing variants/mutants of the polypeptide provided as SEQ ID NO:1 sequence information from related molecules can be used. For example, in an embodiment the polypeptide comprises at least 80%, at least 90%, at least 95% or all of the amino acids conserved between PuhB and PuhA.

Furthermore, if desired, unnatural amino acids or chemical amino acid analogues can be introduced as a substitution or addition into the polypeptides of the present invention. Such amino acids include, but are not limited to, the D-isomers of the common amino acids, 2,4-diaminobutyric acid, α-amino isobutyric acid, 4-aminobutyric acid, 2-aminobutyric acid, 6-amino hexanoic acid, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, β-alanine, fluoro-amino acids, designer amino acids such as β-methyl amino acids, Cα-methyl amino acids, Na-methyl amino acids, and amino acid analogues in general.

Also included within the scope of the invention are polypeptides which are differentially modified during or after synthesis, for example, by biotinylation, benzylation, glycosylation, acetylation, phosphorylation, amidation, derivatization by known protecting/blocking groups, proteolytic cleavage, linkage to an antibody molecule or other cellular ligand, etc. These modifications may serve to increase the stability and/or bioactivity of the polypeptides of the invention.

Polypeptides of the present invention can be produced in a variety of ways, including production and recovery of natural polypeptides, production and recovery of recombinant polypeptides, and chemical synthesis of the polypeptides. In one embodiment, an isolated polypeptide of the present invention is produced by culturing a cell capable of expressing the polypeptide under conditions effective to produce the polypeptide, and recovering the polypeptide. A preferred cell to culture is a host cell of the present invention. Effective culture conditions include, but are not limited to, effective media, bioreactor, temperature, pH and oxygen conditions that permit polypeptide production. An effective medium refers to any medium in which a cell is cultured to produce a polypeptide of the present invention. Such medium typically comprises an aqueous medium having assimilable carbon, nitrogen and phosphate sources, and appropriate salts, minerals, metals and other nutrients, such as vitamins. Cells of the present invention can be cultured in conventional fermentation bioreactors, shake flasks, test tubes, microtiter dishes, and petri plates. Culturing can be carried out at a temperature, pH and oxygen content appropriate for a host cell. Such culturing conditions are within the expertise of one of ordinary skill in the art.

### Polynucleotides and Oligonucleotides

By an “isolated polynucleotide”, including DNA, RNA, or a combination of these, single or double stranded, in the sense or antisense orientation or a combination of both, or otherwise, we mean a polynucleotide which is at least partially separated from the polynucleotide sequences with which it is associated or linked in its native state. Preferably, the isolated polynucleotide is at least 60% free, preferably at least 75% free, and most preferably at least 90% free from other components with which it is naturally associated. Furthermore, the term “polynucleotide” is used interchangeably herein with the term “nucleic acid”.

The term “exogenous” in the context of a polynucleotide refers to the polynucleotide when present in a cell, or in a cell-free expression system, in an altered amount compared to its native state. Preferably, the cell is a cell that does not naturally comprise the polynucleotide. In an alternate embodiment, the cell is a cell which comprises an exogenous polynucleotide resulting in an altered, preferably increased, amount of the polypeptide to be produced. An exogenous polynucleotide of the invention includes polynucleotides which have not been separated from other components of the cell or cell-free expression system, in which it is present, and polynucleotides produced in such cells or cell-free systems which are subsequently purified away from at least some other components. The exogenous polynucleotide can be a contiguous stretch of nucleotides existing in nature, or comprise two or more contiguous stretches of nucleotides from different sources (naturally occurring and/or synthetic) joined to form a single polynucleotide. Typically such chimeric polynucleotides comprise at least an open reading frame encoding a polypeptide of the invention operably linked to a promoter suitable of driving transcription of the open reading frame in a cell of interest.

The % identity of a polynucleotide is determined by GAP (Needleman and Wunsch, 1970) analysis (GCG program) with a gap creation penalty=5, and a gap extension penalty=0.3. Unless stated otherwise, the query sequence is at least 45 nucleotides in length, and the GAP analysis aligns the two sequences over a region of at least 45 nucleotides. Preferably, the query sequence is at least 150 nucleotides in length, and the GAP analysis aligns the two sequences over a region of at least 150 nucleotides. More preferably, the query sequence is at least 300 nucleotides in length and the GAP analysis aligns the two sequences over a region of at least 300 nucleotides. Even more preferably, the GAP analysis aligns the two sequences over their entire length.

With regard to the defined polynucleotides, it will be appreciated that % identity figures higher than those provided above will encompass preferred embodiments. Thus, where applicable, in light of the minimum % identity figures, it is preferred that a polynucleotide of the invention comprises a sequence which is at least 40%, more preferably at least 45%, more preferably at least 50%, more preferably at least 55%, more preferably at least 60%, more preferably at least 65%, more preferably at least 70%, more preferably at least 75%, more preferably at 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO.

Polynucleotides of the invention include those which hybridize under stringent conditions to a nucleic acid encoding SEQ ID NO:1.

As used herein, the term “hybridizes” refers to the ability of two single stranded nucleic acid molecules being able to form at least a partially double stranded nucleic acid through hydrogen bonding.

As used herein, the phrase “stringent conditions” refers to conditions under which a polynucleotide, probe, primer and/or oligonucleotide will hybridize to its target sequence. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e.g., 10 to 50 nucleotides) and at least about 60° C. for longer probes, primers and oligonucleotides. Stringent conditions may also be achieved with the addition of destabilizing agents, such as formamide.

Stringent conditions are known to those skilled in the art and can be found in Ausubel et al. (supra), Current Protocols In Molecular Biology, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions is hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2.×SSC, 0.01% BSA at 50° C.

Polynucleotides of the present invention may possess, when compared to naturally occurring polynucleotides, one or more mutations which are deletions, insertions, and/or substitutions of nucleotide residues. Mutants can be either naturally occurring (that is to say, isolated from a natural source) or synthetic (for example, by performing site-directed mutagenesis on the nucleic acid).

The present invention includes oligonucleotides that can be used as, for example, probes to identify nucleic acid molecules, or primers to produce nucleic acid molecules. Oligonucleotides of the present invention used as probes are typically conjugated with a detectable label such as a radioisotope, an enzyme, biotin, a fluorescent molecule or a chemiluminescent molecule. Probes and/or primers can be used to clone homologues of the polynucleotides of the invention from other species or strains. Furthermore, hybridization techniques known in the art can also be used to screen genomic or cDNA libraries for such homologues.

Oligonucleotides of the present invention can be RNA, DNA, or derivatives of either. Although the terms polynucleotide and oligonucleotide have overlapping meaning, oligonucleotides are typically relatively short single stranded molecules. The minimum size of such oligonucleotides is the size required for the formation of a stable hybrid between an oligonucleotide and a complementary sequence on a target nucleic acid molecule. Preferably, the oligonucleotides are at least 15 nucleotides, more preferably at least 18 nucleotides, more preferably at least 19 nucleotides, more preferably at least 20 nucleotides, even more preferably at least 25 nucleotides in length.

Usually, monomers of a polynucleotide or oligonucleotide are linked by phosphodiester bonds or analogues thereof. Analogs of phosphodiester linkages include: phosphorothioate, phosphorodithioate, phosphoroselenoate, phosphorodiselenoate, phosphoroanilothioate, phosphoranilidate, phosphoramidate.

### Recombinant Vectors

One embodiment of the present invention includes a recombinant vector, which comprises at least one isolated polynucleotide of the present invention, inserted into any vector capable of delivering the polynucleotide into a host cell. Such a vector contains heterologous polynucleotide sequences, that is polynucleotide sequences that are not naturally found adjacent to polynucleotides of the present invention and that preferably are derived from a species other than the species from which the polynucleotides of the present invention are derived. The vector can be either RNA or DNA, either prokaryotic or eukaryotic, and typically is a transposon (such as described in U.S. Pat. No. 5,792,294), a virus, or a plasmid.

One type of recombinant vector comprises a polynucleotide of the present invention operably linked to an expression vector. The phrase “operably linked” refers to insertion of a polynucleotide into an expression vector in a manner such that the polynucleotide is able to be expressed when transformed into a host cell. As used herein, an “expression vector” is a DNA or RNA vector that is capable of transforming a host cell and of effecting expression of a specified polynucleotide. Preferably, the expression vector is also capable of replicating within the host cell. Expression vectors can be either prokaryotic or eukaryotic, and are typically viruses or plasmids. Expression vectors of the present invention include any vectors that function (i.e., direct gene expression) in host cells of the present invention, including in bacterial, fungal, endoparasite, arthropod, animal, and plant cells. Vectors of the invention can also be used to produce the polypeptide in a cell-free expression system, such systems are well known in the art.

“Operably linked” as used herein refers to a functional relationship between two or more nucleic acid (e.g., DNA) segments. Typically, it refers to the functional relationship of a transcriptional regulatory element to a transcribed sequence. For example, a promoter is operably linked to a coding sequence, such as a polynucleotide defined herein, if it stimulates or modulates the transcription of the coding sequence in an appropriate host cell and/or in a cell-free expression system. Generally, promoter transcriptional regulatory elements that are operably linked to a transcribed sequence are physically contiguous to the transcribed sequence, i.e., they are cis-acting. However, some transcriptional regulatory elements, such as enhancers, need not be physically contiguous or located in close proximity to the coding sequences whose transcription they enhance.

In particular, recombinant vectors of the present invention contain regulatory sequences such as transcription control sequences, translation control sequences, origins of replication, and other regulatory sequences that are compatible with the host cell and that control the expression of polynucleotides of the present invention. In particular, recombinant vectors of the present invention include transcription control sequences. Transcription control sequences are sequences which control the initiation, elongation, and termination of transcription. Particularly important transcription control sequences are those which control transcription initiation, such as promoter, enhancer, operator and repressor sequences. Suitable transcription control sequences include any transcription control sequence that can function in at least one of the recombinant cells of the present invention. A variety of such transcription control sequences are known to those skilled in the art. Preferred transcription control sequences include those which function in bacterial, yeast, arthropod, nematode, plant or mammalian cells, such as, but not limited to, tac, lac, trp, trc, oxy-pro, omp/lpp, rrnB, bacteriophage lambda, bacteriophage T7, T7lac, bacteriophage T3, bacteriophage SP6, bacteriophage SP01, metallothionein, alpha-mating factor, Pichia alcohol oxidase, alphavirus subgenomic promoters (such as Sindbis virus subgenomic promoters), antibiotic resistance gene, baculovirus, Heliothis zea insect virus, vaccinia virus, herpesvirus, raccoon poxvirus, other poxvirus, adenovirus, cytomegalovirus (such as intermediate early promoters), simian virus 40, retrovirus, actin, retroviral long terminal repeat, Rous sarcoma virus, heat shock, phosphate and nitrate transcription control sequences, as well as other sequences capable of controlling gene expression in prokaryotic or eukaryotic cells.

Coding sequences of the polypeptides of the invention can be optimized to maximize expression is a particular host cell using known techniques.

### Host and Recombinant Cells

As used herein, the term “host cell” refers to a cell capable of being transformed with an exogenous polynucleotide of the invention. Once transformed, the host cell can be referred to as a “recombinant cell”. The term “recombinant cell” includes direct or indirect progeny cells thereof comprising the polynucleotide. Transformation of a polynucleotide into a host cell can be accomplished by any method by which a polynucleotide can be inserted into the cell. Transformation techniques include, but are not limited to, transfection, electroporation, microinjection, lipofection, adsorption, and protoplast fusion. A recombinant cell may remain unicellular or may grow into a tissue, organ or a multicellular organism. Transformed polynucleotides of the present invention can remain extrachromosomal or can integrate into one or more sites within a chromosome of the transformed (i.e., recombinant) cell in such a manner that their ability to be expressed is retained.

Suitable host cells to transform include any cell that can be transformed with a polynucleotide of the present invention. Host cells of the present invention either can be endogenously (i.e., naturally) capable of producing polypeptides of the present invention or can be capable of producing such polypeptides after being transformed with at least one polynucleotide of the present invention. Host cells of the present invention can be any cell capable of producing at least one protein of the present invention, and include bacterial, fungal (including yeast), parasite, nematode, arthropod, animal and plant cells. Examples of host cells include Salmonella, Escherichia, Bacillus, Listeria, Saccharomyces, Spodoptera, Mycobacteria, Trichoplusia, BHK (baby hamster kidney) cells, MDCK cells, CRFK cells, CV-1 cells, COS (e.g., COS-7) cells, and Vero cells. Further examples of host cells are E. coli, including E coli K-12 derivatives; Salmonella typhi; Salmonella typhimurium, including attenuated strains; Spodoptera frugiperda; Trichoplusia ni; and non-tumorigenic mouse myoblast G8 cells (e.g., ATCC CRL 1246). Particularly preferred host cells are plant cells.

Recombinant DNA technologies can be used to improve expression of an exogenous polynucleotide by manipulating, for example, the number of copies of the polynucleotide molecule within a host cell, the efficiency with which those polynucleotide molecules are transcribed, the efficiency with which the resultant transcripts are translated, and the efficiency of post-translational modifications. Recombinant techniques useful for increasing the expression of polynucleotides of the present invention include, but are not limited to, operatively linking polynucleotides to high-copy number plasmids, integration of the polynucleotide into one or more host cell chromosomes, addition of vector stability sequences to plasmids, substitutions or modifications of transcription control signals (e.g., promoters, operators, enhancers), substitutions or modifications of translational control signals (e.g., ribosome binding sites, Shine-Dalgarno sequences), modification of polynucleotides of the present invention to correspond to the codon usage of the host cell, and the deletion of sequences that destabilize transcripts.

### Transgenic Plants

The term “plant” as used herein refers to whole plants, such as, for example, a plant growing in a field, and any substance which is present in, obtained from, derived from, or related to a plant, such as, for example, vegetative structures (e.g., leaves, stems), roots, floral organs/structures, seeds (including embryo, endosperm, and seed coat), plant tissue (e.g., vascular, tissue, ground tissue, and the like), cells (e.g., pollen), and progeny of same.

Plants contemplated for use in the practice of the present invention include both monocotyledons and dicotyledons. Target plants include, but are not limited to, the following: cereals (wheat, barley, rye, oats, rice, sorghum, triticale, and related crops); beet (sugar beet and fodder beet); pomes (apples, pears), stone fruit (plums, peaches, almonds, cherries) tropical fruit (bananas, pineapple, pawpaws) and soft fruit (cherries, strawberries, raspberries and black-berries); leguminous plants (beans, lentils, peas, soybeans, lucerne, lupins); oil plants (rape, mustard, poppy, olives, sunflowers, coconut, castor oil plants, cocoa beans, groundnuts); cucumber plants (marrows, cucumbers, melons); fibre plants (cotton, cotton defoliant, flax, hemp, jute); citrus fruit (oranges, lemons, grapefruit, mandarins); vegetables (spinach, lettuce, asparagus, cabbages, carrots, onions, tomatoes, potatoes, paprika); lauraceae (avocados, cinnamon, camphor); or plants such as maize, tobacco, nuts, coffee, sugar cane, tea, vines, hops, turf including perennial grass and the phalarsis cultivars sirolan and sirone, and natural rubber plants, as well as ornamentals (flowers such as daffodils, gladioli and tulips, shrubs such as Duboisia, broad-leaved trees and evergreens, such as conifers). Preferably, the plants are angiosperms.

Transgenic plants, as defined in the context of the present invention include plants (as well as parts and cells of said plants) and their progeny which have been genetically modified using recombinant techniques to cause production of at least one polypeptide of the present invention in the desired plant or plant organ. Transgenic plants can be produced using techniques known in the art, such as those generally described in A. Slater et al., Plant Biotechnology—The Genetic Manipulation of Plants, Oxford University Press (2003); and P. Christou and H. Klee, Handbook of Plant Biotechnology, John Wiley and Sons (2004).

A “transgenic plant” refers to a plant that contains a gene construct (“transgene”) not found in a wild-type plant of the same species, variety or cultivar. A “transgene” as referred to herein, has the normal meaning in the art of biotechnology and includes an exogenous polynucleotide sequence which has been produced or altered by recombinant DNA or RNA technology and which has been introduced into the plant cell. The transgene may include polynucelotide sequences derived from a plant cell. Typically, the transgene has been introduced into the plant by human manipulation such as, for example, by transformation but any method can be used as one of skill in the art recognizes.

In a preferred embodiment of the invention, the transgenic plants are homozygous for each and every gene that has been introduced (transgene) so that their progeny do not segregate for the desired phenotype. The transgenic plants may also be heterozygous for the introduced transgene(s), such as, for example, in F1 progeny which have been grown from hybrid seed. Such plants may provide advantages such as hybrid vigour, well known in the art.

A polynucleotide of the present invention may be expressed constitutively in the transgenic plants during all stages of development. Depending on the use of the plant or plant organs, the polypeptides may be expressed in a stage-specific manner. Furthermore, the polynucleotides may be expressed tissue-specifically.

Regulatory sequences which are known or are found to cause expression of a gene encoding a polypeptide of interest in plants may be used in the present invention. The choice of the regulatory sequences used depends on the target plant and/or target organ of interest. Such regulatory sequences may be obtained from plants or plant viruses, or may be chemically synthesized. Such regulatory sequences are well known to those skilled in the art.

A number of vectors suitable for stable transfection of plant cells or for the establishment of transgenic plants have been described in, for example, Pouwels et al., Cloning Vectors: A Laboratory Manual (1985, supp. 1987); Weissbach and Weissbach, Methods for Plant Molecular Biology, Academic Press (1989); and Gelvin et al., Plant Molecular Biology Manual, Kluwer Academic Publishers (1990). Typically, plant expression vectors include, for example, one or more cloned plant genes under the transcriptional control of 5′ and 3′ regulatory sequences and a dominant selectable marker. Such plant expression vectors also can contain a promoter regulatory region (e.g., a regulatory region controlling inducible or constitutive, environmentally- or developmentally-regulated, or cell- or tissue-specific expression), a transcription initiation start site, a ribosome binding site, an RNA processing signal, a transcription termination site, and/or a polyadenylation signal.

A number of constitutive promoters that are active in plant cells have been described. Suitable promoters for constitutive expression in plants include, but are not limited to, the cauliflower mosaic virus (CaMV) 35S promoter, the Figwort mosaic virus (FMV) 35S, the sugarcane bacilliform virus promoter, the commelina yellow mottle virus promoter, the light-inducible promoter from the small subunit of the ribulose-1,5-bis-phosphate carboxylase, the rice cytosolic triosephosphate isomerase promoter, the adenine phosphoribosyltransferase promoter of Arabidopsis, the rice actin 1 gene promoter, the mannopine synthase and octopine synthase promoters, the Adh promoter, the sucrose synthase promoter, the R gene complex promoter, and the chlorophyll α/β binding protein gene promoter. These promoters have been used to create DNA vectors that have been expressed in plants; see for example, PCT publication WO 84/02913. All of these promoters have been used to create various types of plant-expressible recombinant DNA vectors.

For the purpose of expression in source tissues of the plant, such as the leaf, seed, root or stem, it is preferred that the promoters utilized in the present invention have relatively high expression in these specific tissues. For this purpose, one may choose from a number of promoters for genes with tissue- or cell-specific or -enhanced expression. Examples of such promoters reported in the literature include the chloroplast glutamine synthetase GS2 promoter from pea, the chloroplast fructose-1,6-biphosphatase promoter from wheat, the nuclear photosynthetic ST-LS1 promoter from potato, the serine/threonine kinase promoter and the glucoamylase (CHS) promoter from Arabidopsis thaliana. Also reported to be active in photosynthetically active tissues are the ribulose-1,5-bisphosphate carboxylase promoter from eastern larch (Larix laricina), the promoter for the Cab gene, Cab6, from pine, the promoter for the Cab-1 gene from wheat, the promoter for the Cab-1 gene from spinach, the promoter for the Cab 1R gene from rice, the pyruvate, orthophosphate dikinase (PPDK) promoter from Zea mays, the promoter for the tobacco Lhcb1*2 gene, the Arabidopsis thaliana Suc2 sucrose-H³⁰ symporter promoter, and the promoter for the thylakoid membrane protein genes from spinach (PsaD, PsaF, PsaE, PC, FNR, AtpC, AtpD, Cab, RbcS).

Other promoters for the chlorophyll α/β-binding proteins may also be utilized in the present invention, such as the promoters for LhcB gene and PsbP gene from white mustard (Sinapis alba). A variety of plant gene promoters that are regulated in response to environmental, hormonal, chemical, and/or developmental signals, can also be used for expression of RNA-binding protein genes in plant cells, including promoters regulated by (1) heat, (2) light (e.g., pea RbcS-3A promoter, maize RbcS promoter); (3) hormones, such as abscisic acid, (4) wounding (e.g., WunI); or (5) chemicals, such as methyl jasminate, salicylic acid, steroid hormones, alcohol, Safeners (see WO 97/06269), or it may also be advantageous to employ (6) organ-specific promoters.

For the purpose of expression in sink tissues of the plant, such as the tuber of the potato plant, the fruit of tomato, or the seed of soybean, canola, cotton, Zea mays, wheat, rice, and barley, it is preferred that the promoters utilized in the present invention have relatively high expression in these specific tissues. A number of promoters for genes with tuber-specific or -enhanced expression are known, including the class I patatin promoter, the promoter for the potato tuber ADPGPP genes, both the large and small subunits, the sucrose synthase promoter, the promoter for the major tuber proteins including the 22 kD protein complexes and proteinase inhibitors, the promoter for the granule bound starch synthase gene (GBSS), and other class I and II patatins promoters. Other promoters can also be used to express a protein in specific tissues, such as seeds or fruits. The promoter for β-conglycinin or other seed-specific promoters such as the napin and phaseolin promoters, can be used. A particularly preferred promoter for Zea mays endosperm expression is the promoter for the glutelin gene from rice, more particularly the Osgt-1 promoter. Examples of promoters suitable for expression in wheat include those promoters for the ADPglucose pyrosynthase (ADPGPP) subunits, the granule bound and other starch synthase, the branching and debranching enzymes, the embryogenesis-abundant proteins, the gliadins, and the glutenins. Examples of such promoters for barley include those for the ADPGPP subunits, the granule bound and other starch synthase, the branching enzymes, the debranching enzymes, sucrose synthases, the hordeins, the embryo globulins, and the aleurone specific proteins.

Root specific promoters may also be used. An example of such a promoter is the promoter for the acid chitinase gene. Expression in root tissue could also be accomplished by utilizing the root specific subdomains of the CaMV 35S promoter that have been identified.

The 5′ non-translated leader sequence can be derived from the promoter selected to express the heterologous gene sequence of the polynucleotide of the present invention, and can be specifically modified if desired so as to increase translation of mRNA. For a review of optimizing expression of transgenes, see Koziel et al. (1996). The 5′ non-translated regions can also be obtained from plant viral RNAs (tobacco mosaic virus, tobacco etch virus, maize dwarf mosaic virus, alfalfa mosaic virus, among others) from suitable eukaryotic genes, plant genes (wheat and maize chlorophyll a/b binding protein gene leader), or from a synthetic gene sequence. The present invention is not limited to constructs wherein the non-translated region is derived from the 5′ non-translated sequence that accompanies the promoter sequence. The leader sequence could also be derived from an unrelated promoter or coding sequence. Leader sequences useful in context of the present invention comprise the maize Hsp70 leader (see U.S. Pat. No. 5,362,865 and U.S. Pat. No. 5,859,347), and the TMV omega element.

The termination of transcription is accomplished by a 3′ non-translated DNA sequence operably linked in the chimeric vector to the polynucleotide of interest. The 3′ non-translated region of a recombinant DNA molecule contains a polyadenylation signal that functions in plants to cause the addition of adenylate nucleotides to the 3′ end of the RNA. The 3′ non-translated region can be obtained from various genes that are expressed in plant cells. The nopaline synthase 3′ untranslated region, the 3′ untranslated region from pea small subunit Rubisco gene, the 3′ untranslated region from soybean 7S seed storage protein gene are commonly used in this capacity. The 3′ transcribed, non-translated regions containing the polyadenylate signal of Agrobacterium tumor-inducing (Ti) plasmid genes are also suitable.

Four general methods for direct delivery of a gene into cells have been described: (1) chemical methods (Graham et al., 1973); (2) physical methods such as microinjection (Capecchi, 1980); electroporation (see WO 87/06614, U.S. Pat. Nos. 5,472,869, 5,384,253, WO 92/09696 and WO 93/21335); and the gene gun (see U.S. Pat. No. 4,945,050 and U.S. Pat. No. 5,141,131); (3) viral vectors (Clapp, 1993; Lu et al., 1993; Eglitis et al., 1988); and (4) receptor-mediated mechanisms (Curiel et al., 1992; Wagner et al., 1992).

Acceleration methods that may be used include, for example, microprojectile bombardment and the like. One example of a method for delivering transforming nucleic acids to plant cells is microprojectile bombardment. This method has been reviewed by Yang et al., Particle Bombardment Technology for Gene Transfer, Oxford Press, Oxford, England (1994). Non-biological particles (microprojectiles) may be coated with nucleic acids and delivered into cells by a propelling force. Exemplary particles include those comprised of tungsten, gold, platinum, and the like. A particular advantage of microprojectile bombardment, in addition to it being an effective means of reproducibly transforming monocots, is that ₙeither the isolation of protoplasts, nor the susceptibility of Agrobacterium infection are required. An illustrative embodiment of a method for delivering DNA into Zea mays cells by acceleration is a biolistics α-particle delivery system that can be used to propel particles coated with DNA through a screen, such as a stainless steel or Nytex screen, onto a filter surface covered with corn cells cultured in suspension. A particle delivery system suitable for use with the present invention is the helium acceleration PDS-1000/He gun which is available from Bio-Rad Laboratories.

For the bombardment, cells in suspension may be concentrated on filters. Filters containing the cells to be bombarded are positioned at an appropriate distance below the microprojectile stopping plate. If desired, one or more screens are also positioned between the gun and the cells to be bombarded.

Alternatively, immature embryos or other target cells may be arranged on solid culture medium. The cells to be bombarded are positioned at an appropriate distance below the microprojectile stopping plate. If desired, one or more screens are also positioned between the acceleration device and the cells to be bombarded. Through the use of techniques set forth herein, one may obtain up to 1000 or more foci of cells transiently expressing a marker gene. The number of cells in a focus that express the exogenous gene product 48 hours post-bombardment often ranges from 1 to 10 and on average, 1 to 3.

In bombardment transformation, one may optimize the pre-bombardment culturing conditions and the bombardment parameters to yield the maximum numbers of stable transformants. Both the physical and biological parameters for bombardment are important in this technology. Physical factors are those that involve manipulating the DNA/microprojectile precipitate or those that affect the flight and velocity of either the macro- or microprojectiles. Biological factors include all steps involved in manipulation of cells before and immediately after bombardment, the osmotic adjustment of target cells to help alleviate the trauma associated with bombardment, and also the nature of the transforming DNA, such as linearized DNA or intact supercoiled plasmids. It is believed that pre-bombardment manipulations are especially important for successful transformation of immature embryos.

In another alternative embodiment, plastids can be stably transformed. Methods disclosed for plastid transformation in higher plants include particle gun delivery of DNA containing a selectable marker and targeting of the DNA to the plastid genome through homologous recombination (see U.S. Pat. No. 5,451,513, U.S. Pat. No. 5,545,818, U.S. Pat. No. 5,877,402, U.S. Pat. No. 5,932479, and WO 99/05265).

Accordingly, it is contemplated that one may wish to adjust various aspects of the bombardment parameters in small scale studies to fully optimize the conditions. One may particularly wish to adjust physical parameters such as gap distance, flight distance, tissue distance, and helium pressure. One may also minimize the trauma reduction factors by modifying conditions that influence the physiological state of the recipient cells and that may therefore influence transformation and integration efficiencies. For example, the osmotic state, tissue hydration and the subculture stage or cell cycle of the recipient cells may be adjusted for optimum transformation. The execution of other routine adjustments will be known to those of skill in the art in light of the present disclosure.

Agrobacterium-mediated transfer is a widely applicable system for introducing genes into plant cells because the DNA can be introduced into whole plant tissues, thereby bypassing the need for regeneration of an intact plant from a protoplast. The use of Agrobacterium-mediated plant integrating vectors to introduce DNA into plant cells is well known in the art (see U.S. Pat. No. 5,177,010, U.S. Pat. No. 5,104,310, U.S. Pat. No. 5,004,863, U.S. Pat. No. 5,159,135). Further, the integration of the T-DNA is a relatively precise process resulting in few rearrangements. The region of DNA to be transferred is defined by the border sequences, and intervening DNA is usually inserted into the plant genome.

Modern Agrobacterium transformation vectors are capable of replication in E. coli as well as Agrobacterium, allowing for convenient manipulations as described in Klee et al., Plant DNA Infectious Agents, Hohn and Schell (editors), Springer-Verlag, New York (1985), pp. 179-203. Moreover, technological advances in vectors for Agrobacterium-mediated gene transfer have improved the arrangement of genes and restriction sites in the vectors to facilitate construction of vectors capable of expressing various polypeptide coding genes. The vectors described have convenient multi-linker regions flanked by a promoter and a polyadenylation site for direct expression of inserted polypeptide coding genes and are suitable for present purposes. In addition, Agrobacterium containing both armed and disarmed Ti genes can be used for the transformations. In those plant varieties where Agrobacterium-mediated transformation is efficient, it is the method of choice because of the facile and defined nature of the gene transfer.

A transgenic plant formed using Agrobacterium transformation methods typically contains a single genetic locus on one chromosome. Such transgenic plants can be referred to as being hemizygous for the added gene. More preferred is a transgenic plant that is homozygous for the added structural gene, that is, a transgenic plant that contains two added genes, one gene at the same locus on each chromosome of a chromosome pair. A homozygous transgenic plant can be obtained by sexually mating (selfing) an independent segregant transgenic plant that contains a single added gene, germinating some of the seed produced and analyzing the resulting plants for the gene of interest.

It is also to be understood that two different transgenic plants can also be mated to produce offspring that contain two independently segregating exogenous genes. Selfing of appropriate progeny can produce plants that are homozygous for both exogenous genes. Back-crossing to a parental plant and out-crossing with a non-transgenic plant are also contemplated, as is vegetative propagation. Descriptions of other breeding methods that are commonly used for different traits and crops can be found in Fehr, Breeding Methods for Cultivar Development, J. Wilcox (editor), American Society of Agronomy, Madison Wis. (1987).

Transformation of plant protoplasts can be achieved using methods based on calcium phosphate precipitation, polyethylene glycol treatment, electroporation, and combinations of these treatments. Application of these systems to different plant varieties depends upon the ability to regenerate that particular plant strain from protoplasts. Illustrative methods for the regeneration of cereals from protoplasts are described (Fujimura et al., 1985; Toriyama et al., 1986; Abdullah et al., 1986).

Other methods of cell transformation can also be used and include but are not limited to introduction of DNA into plants by direct DNA transfer into pollen, by direct injection of DNA into reproductive organs of a plant, or by direct injection of DNA into the cells of immature embryos followed by the rehydration of desiccated embryos.

The regeneration, development, and cultivation of plants from single plant protoplast transformants or from various transformed explants is well known in the art (Weissbach et al., Methods for Plant Molecular Biology, Academic Press, San Diego, Calif. (1988). This regeneration and growth process typically includes the steps of selection of transformed cells, culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil.

The development or regeneration of plants containing the foreign, exogenous gene is well known in the art. Preferably, the regenerated plants are self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. A transgenic plant of the present invention containing a desired exogenous nucleic acid is cultivated using methods well known to one skilled in the art.

Methods for transforming dicotyledons, primarily by use of Agrobacterium tumefaciens, and obtaining transgenic plants have been published for cotton (U.S. Pat. No. 5,004,863, U.S. Pat. No. 5,159,135, U.S. Pat. No. 5,518,908); soybean (U.S. Pat. No. 5,569,834, U.S. Pat. No. 5,416,011); Brassica (U.S. Pat. No. 5,463,174); peanut (Cheng et al., 1996); and pea (Grant et al., 1995).

Methods for transformation of cereal plants such as wheat and barley for introducing genetic variation into the plant by introduction of an exogenous nucleic acid and for regeneration of plants from protoplasts or immature plant embryos are well known in the art, see for example, CA 2,092,588, AU 61781/94, AU 667939, U.S. Pat. No. 6,100,447, PCT/US97/10621, U.S. Pat. No. 5,589,617, U.S. Pat. No. 6,541,257, and other methods are set out in WO 99/14314. Preferably, transgenic wheat or barley plants are produced by Agrobacterium tumefaciens mediated transformation procedures. Vectors carrying the desired nucleic acid construct may be introduced into regenerable wheat cells of tissue cultured plants or explants, or suitable plant systems such as protoplasts.

The regenerable wheat cells are preferably from the scutellum of immature embryos, mature embryos, callus derived from these, or the meristematic tissue.

To confirm the presence of the transgenes in transgenic cells and plants, a polymerase chain reaction (PCR) amplification or Southern blot analysis can be performed using methods known to those skilled in the art. Expression products of the transgenes can be detected in any of a variety of ways, depending upon the nature of the product, and include Western blot and enzyme assay. One particularly useful way to quantitate protein expression and to detect replication in different plant tissues is to use a reporter gene, such as GUS. Once transgenic plants have been obtained, they may be grown to produce plant tissues or parts having the desired phenotype. The plant tissue or plant parts, may be harvested, and/or the seed collected. The seed may serve as a source for growing additional plants with tissues or parts having the desired characteristics.

Transgenic plants of the invention may comprise further transgenes beyond those of the invention which enhance the plants tolerance/resistance to a phenylurea, carbamate, and/or organophosphate.

### Transgenic Non-Human Animals

A “transgenic non-human animal” refers to an animal, other than a human, that contains a gene construct (“transgene”) not found in a wild-type animal of the same species or breed. A “transgene” as referred to herein, has the normal meaning in the art of biotechnology and includes an exogenous polynucleotide sequence which has been produced or altered by recombinant DNA or RNA technology and which has been introduced into an animal cell. The transgene may include polynucleotide sequences derived from an animal cell. Typically, the transgene has been introduced into the animal by human manipulation such as, for example, by transformation but any method can be used as one of skill in the art recognizes.

Techniques for producing transgenic animals are well known in the art. A useful general textbook on this subject is Houdebine, Transgenic animals—Generation and Use, Harwood Academic (1997).

Heterologous DNA can be introduced, for example, into fertilized mammalian ova. For instance, totipotent or pluripotent stem cells can be transformed by microinjection, calcium phosphate mediated precipitation, liposome fusion, retroviral infection or other means. The transformed cells are then introduced into the embryo, and the embryo then develops into a transgenic animal. In a highly preferred method, developing embryos are infected with a retrovirus containing the desired DNA, and transgenic animals produced from the infected embryo. In a most preferred method, however, the appropriate DNAs are coinjected into the pronucleus or cytoplasm of embryos, preferably at the single cell stage, and the embryos allowed to develop into mature transgenic animals.

Another method used to produce a transgenic animal involves microinjecting a nucleic acid into pro-nuclear stage eggs by standard methods. Injected eggs are then cultured before transfer into the oviducts of pseudopregnant recipients.

Transgenic animals may also be produced by nuclear transfer technology. Using this method, fibroblasts from donor animals are stably transfected with a plasmid incorporating the coding sequences for a binding domain or binding partner of interest under the control of regulatory sequences. Stable transfectants are then fused to enucleated oocytes, cultured and transferred into female recipients.

### Compositions

Compositions of the present invention can include excipients, also referred to herein as “acceptable carriers”. An excipient can be any material that the animal, plant, plant or animal material, or environment (including soil and water samples) to be treated can tolerate. Examples of such excipients include water, saline, Ringer's solution, dextrose solution, Hank's solution, and other aqueous physiologically balanced salt solutions. Nonaqueous vehicles, such as fixed oils, sesame oil, ethyl oleate, or triglycerides may also be used. Other useful formulations include suspensions containing viscosity enhancing agents, such as sodium carboxymethylcellulose, sorbitol, or dextran. Excipients can also contain minor amounts of additives, such as substances that enhance isotonicity and chemical stability. Examples of buffers include phosphate buffer, bicarbonate buffer and Tris buffer, while examples of preservatives include thimerosal or o-cresol, formalin and benzyl alcohol. Excipients can also be used to increase the half-life of a composition, for example, but are not limited to, polymeric controlled release vehicles, biodegradable implants, liposomes, bacteria, viruses, other cells, oils, esters, and glycols.

Furthermore, a polypeptide described herein can be provided in a composition that enhances the rate and/or degree of hydrolysis of a phenylurea, carbamate, and/or organophosphate, or increases the stability of the polypeptide. For example, the polypeptide can be immobilized on a polyurethane matrix (Gordon et al., 1999), or encapsulated in appropriate liposomes (Petrikovics et al., 2000a and b). The polypeptide can also be incorporated into a composition comprising a foam, such as those used routinely in fire-fighting (LeJeune et al., 1998).

One embodiment of the present invention is a controlled release formulation that is capable of slowly releasing a composition of the present invention into an animal, plant, animal or plant material, or the environment (including soil and water samples). As used herein, a “controlled release formulation” comprises a composition of the present invention in a controlled release vehicle. Suitable controlled release vehicles include, but are not limited to, biocompatible polymers, other polymeric matrices, capsules, microcapsules, microparticles, bolus preparations, osmotic pumps, diffusion devices, liposomes, lipospheres, and transdermal delivery systems. Preferred controlled release formulations are biodegradable (i.e., bioerodible).

A preferred controlled release formulation of the present invention is capable of releasing a composition of the present invention into soil or water which is in an area comprising a phenylurea, carbamate, and/or organophosphate, particularly diuron. The formulation is preferably released over a period of time ranging from about 1 to about 12 months. A preferred controlled release formulation of the present invention is capable of effecting a treatment preferably for at least about 1 month, more preferably for at least about 3 months, even more preferably for at least about 6 months, even more preferably for at least about 9 months, and even more preferably for at least about 12 months.

The concentration of the polypeptide, vector, bacteria, extract, or host cell etc., of the present invention that will be required to produce effective compositions for hydrolysing a phenylurea, carbamate, and/or organophosphate will depend on the nature of the sample to be decontaminated, the concentration of the phenylurea, carbamate, and/or organophosphate in the sample, and the formulation of the composition. The effective concentration of the polypeptide, vector, bacteria, extract, or host cell etc., within the composition can readily be determined experimentally, as will be understood by the skilled person.

Enzymes of the invention, and/or microorganisms encoding therefor, can be used in coating compositions as generally described in WO 2004/112482 and WO 2005/26269.

### Antibodies

The term “antibody” as used in this invention includes polyclonal antibodies, monoclonal antibodies, bispecific antibodies, diabodies, triabodies, heteroconjugate antibodies, chimeric antibodies including intact molecules as well as fragments thereof, such as Fab, F(ab′)₂, and Fv which are capable of binding the epitopic determinant, and other antibody-like molecules.

Antibody fragments retain some ability to selectively bind with its antigen or receptor and are defined as follows:

(1) Fab, the fragment which contains a monovalent antigen-binding fragment of an antibody molecule can be produced by digestion of whole antibody with the enzyme papain to yield an intact light chain and a portion of one heavy chain;

(2) Fab′, the fragment of an antibody molecule can be obtained by treating whole antibody with pepsin, followed by reduction, to yield an intact light chain and a portion of the heavy chain; two Fab' fragments are obtained per antibody molecule;

(3) (Fab′)₂, the fragment of the antibody that can be obtained by treating whole antibody with the enzyme pepsin without subsequent reduction; F(ab)2 is a dimer of two Fab' fragments held together by two disulfide bonds;

(4) Fv, defined as a genetically engineered fragment containing the variable region of the light chain and the variable region of the heavy chain expressed as two chains; and

(5) Single chain antibody (“SCA”), defined as a genetically engineered molecule containing the variable region of the light chain, the variable region of the heavy chain, linked by a suitable polypeptide linker as a genetically fused single chain molecule.

Methods of making these fragments are known in the art (see for example, Harlow and Lane, Antibodies: A Laboratory Manual, Cold Spring Harbor Laboratory, New York (1988)).

(6) Single domain antibody, typically a variable heavy domain devoid of a light chain.

The term “specifically binds” refers to the ability of the antibody to bind to at least one polypeptide of the present invention but not other known proteins, in particular not PuhA provided as SEQ ID NO:3.

As used herein, the term “epitope” refers to a region of a polypeptide of the invention which is bound by the antibody. An epitope can be administered to an animal to generate antibodies against the epitope, however, antibodies of the present invention preferably specifically bind the epitope region in the context of the entire polypeptide.

If polyclonal antibodies are desired, a selected mammal (e.g., mouse, rabbit, goat, horse, etc.) is immunised with an immunogenic polypeptide of the invention. Serum from the immunised animal is collected and treated according to known procedures. If serum containing polyclonal antibodies contains antibodies to other antigens, the polyclonal antibodies can be purified by immunoaffinity chromatography. Techniques for producing and processing polyclonal antisera are known in the art. In order that such antibodies may be made, the invention also provides polypeptides of the invention or fragments thereof haptenised to another polypeptide for use as immunogens in animals.

Monoclonal antibodies directed against polypeptides of the invention can also be readily produced by one skilled in the art. The general methodology for making monoclonal antibodies by hybridomas is well known. Immortal antibody-producing cell lines can be created by cell fusion, and also by other techniques such as direct transformation of B lymphocytes with oncogenic DNA, or transfection with Epstein-Barr virus. Panels of monoclonal antibodies produced can be screened for various properties; i.e., for isotype and epitope affinity.

An alternative technique involves screening phage display libraries where, for example the phage express scFv fragments on the surface of their coat with a large variety of complementarity determining regions (CDRs). This technique is well known in the art.

Other techniques for producing antibodies of the invention are known in the art.

Antibodies of the invention may be bound to a solid support and/or packaged into kits in a suitable container along with suitable reagents, controls, instructions and the like.

In an embodiment, antibodies of the present invention are detectably labeled. Exemplary detectable labels that allow for direct measurement of antibody binding include radiolabels, fluorophores, dyes, magnetic beads, chemiluminescers, colloidal particles, and the like. Examples of labels which permit indirect measurement of binding include enzymes where the substrate may provide for a coloured or fluorescent product. Additional exemplary detectable labels include covalently bound enzymes capable of providing a detectable product signal after addition of suitable substrate. Examples of suitable enzymes for use in conjugates include horseradish peroxidase, alkaline phosphatase, malate dehydrogenase and the like. Where not commercially available, such antibody-enzyme conjugates are readily produced by techniques known to those skilled in the art. Further, exemplary detectable labels include biotin, which binds with high affinity to avidin or streptavidin; fluorochromes (e.g., phycobiliproteins, phycoerythrin and allophycocyanins; fluorescein and Texas red), which can be used with a fluorescence activated cell sorter; haptens; and the like. Preferably, the detectable label allows for direct measurement in a plate luminometer, for example, biotin. Such labeled antibodies can be used in techniques known in the art to detect polypeptides of the invention.

### Micro-organism Deposit Details

Mycobacterium brisbanense JK1 was deposited on 16 May 2008 with the National Measurement Institute, 51-65 Clarke Street, South Melbourne, Victoria 3205, Australia under accession number V08/013277.

This deposit was made under the provisions of the Budapest Treaty on the International Recognition of the Deposit of Microorganisms for the Purpose of Patent Procedure and the Regulations thereunder. This assures maintenance of viable cultures for 30 years from the date of deposit. The organisms will be made available by the National Measurement Institute under the terms of the Budapest Treaty which assures permanent and unrestricted availability of the progeny of the culture to the public upon issuance of the pertinent patent.

The assignee of the present application has agreed that if the culture deposit should die or be lost or destroyed when cultivated under suitable conditions, it will be promptly replaced on notification with a viable specimen of the same culture.

Availability of a deposited strain is not to be construed as a license to practice the invention in contravention of the rights granted under the authority of any government in accordance with its patent laws.

### EXAMPLES

### Example 1

### Materials and Methods

### Bacterial Strains and Media

Difco™ nutrient broth, Pseudomonas agar and nutrient agar were purchased from Becton, Dickinson and Co. Luria broth (LB), LB agar, tris-acetate-EDTA (TAE), tris-EDTA (TE) and antibiotics (hygromycin, ampicillin, kanamycin and chloramphenicol) were prepared as described in Sambrook et al. (1989). Minimal media (MM) was prepared as described by Sorensen and Aamand (2003). All pesticides and their metabolites were of the highest available purity (>97%). Carbaryl and parathion were obtained from Chem Service, and other pesticides were purchased from Sigma. The N, N-dimethyl, O-4-nitrophenyl carbamate was a gift from Dr Timothy Bugg, University of Warwick, England. Details of the linuron ester and 2-dimethylamino-5,6-dimethyl-4-hydroxy pyrimidine (DDHP) synthesis are shown below.

Soil samples were provided by John Reghenzani (BSES Ltd.) from sugar cane growing areas of Queensland where diuron had been applied. Soil samples (1 g) were suspended in 50 mL of MM in 250 mL Erlenmeyer flasks supplemented with 10-50 ppm diuron in the presence of a mixture of glucose, glycerol and succinate (10 mM each) as carbon sources as required. All enrichment cultures were grown at 28° C. in the dark. Subculturing involved transfer of 0.2-4% of the culture volume into fresh MM, supplemented appropriately. Degradation was observed by extracting metabolites from 500 μL of culture using 500 μL of acetonitrile, prior to analysis by high performance liquid chromatograpy (HPLC). Once a stable, diuron-degrading culture was established, individual bacterial strains were isolated by dilution plating on LB agar, nutrient agar (Difco™) and Pseudomonas agar (Difco™). Individual colonies were picked and used to inoculate further flasks of appropriately supplemented MM.

Escherichia coli LE392MP (Epicentre), JM109 (Promega) or Electro-10 blue (Stratagene) and Mycobacterium smegmatis mc2 clones were routinely grown at 37° C. on LB agar or in liquid media using appropriate antibiotics. Electrocompetant M. smegmatis mc2 cells were prepared as described elsewhere (Jacobs et al., 1991). Transformations of E. coli and M. smegmatis were carried out by electroporation with 0.1-1 μg of DNA using a Biorad genepulser II at 200 Ω, 2500 V and 2.5 μF with disposable electroporation curvettes (BTX-Harvard apparatus). Post electroporation cells were grown-out at 37° C. for a period just less than the doubling time in 500 μL LB, followed by plating on solid media containing the appropriate antibiotic. Tween 80 (0.05% v/v) was added to M. smegmatis transformations to ensure cells did not clump. Arthrobacter globiformis D47 containing the plasmid pHRIM620, which confers the diuron degrading phenotype, was cultured in 1 L flasks containing LB supplemented with 0.05 mg.mL⁻¹ sulfamethoxazole and 0.001 mg.mL⁻¹ trimethoprim at 28 ° C. for 23 hours. M. brisbanense JK1 was cultured for 2 days at 28° C. in 3 times 1 L of LB containing 0.05% tween 80.

### Synthesis of 3,4-dichlorophenyl N-methoxy-N-methylcarbamate (the linuron ester)

The method followed was that used for 3-dimethylaminophenyl N-methoxy-N-methylcarbamate (compound XIV) in Wustner et al. (1978):

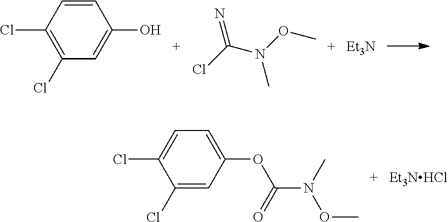

1.63 g 3,4-dichlorophenol (10 mmol, Aldrich) was weighed into a 100 ml round bottomed, septum-sealed flask, flushed with nitrogen and dissolved in 15 ml benzene (AR). 1.53 ml (11 mmol) distilled triethylamine was added and the resulting solution cooled in an ice bath. 1.36 g N-methoxy-N-methylcarbamoyl chloride (11 mmol, Acros Organics) was weighed into a 10 ml round bottomed, septum-sealed flask, flushed with nitrogen and dissolved in 5 ml benzene. This solution was added over 10 minutes to the ice-cooled dichlorophenol/triethylamine solution; a white precipitate (triethylamine hydrochloride) was observed to separate during the addition. The ice bath was removed after 1 hour and the reaction mixture stirred at room temperature for a further 5 hours. 20 ml ice-water was then added to quench the reaction, the organic phase separated and washed with 5 ml 0.5 M aqueous hydrochloric acid followed by three 5 ml portions of saturated aqueous brine. The benzene solution was dried with anhydrous sodium sulfate, filtered and the drying agent washed with 5 portions of benzene. The filtrate and washings were combined and rotary evaporated to give 2.53 g of a mobile oil with a slight yellow tinge.

The crude product was flash chromatographed on a 5 cm diameter, 10 cm bed of 230-400 mesh silica (Carlo Erba SDS), eluted with distilled chloroform, collecting twenty 50 ml fractions. Fractions 5-14 were pooled and rotary evaporated. The residual oil was dissolved in distilled chloroform filtered through a 25 mm diameter 0.45 μm pore PVDF membrane syringe filter into a weighed 100 ml round bottomed flask. The solvent was removed by rotary evaporation to give a colourless oil which was placed under high vacuum overnight.

Yield 2.36 g 94%.

¹H NMR (CDCl₃ 300 MHz): 3.28 ppm, s, 3H, CH₃N; 3.59 ppm, s, 3H, CH₃O; 7.04 ppm, dd J 8.8, 2.8 Hz, 1H, H-6; 7.31 ppm, d, J2.8 Hz, 1H, H-2; 7.44 ppm, d, J8.8 Hz, 1H, H-5.

¹³CNMR (CDCl₃, 75 MHz): 35.4 ppm CH₃N; 61.7 ppm, CH₃O; 121.2, 123.8, 129.3, 130.5, 132.6, 149.4 ppm, Aromatic C; 154.0 ppm, C═O.

EIMS: m/z, intensity: 253, 5, M⁺ (³⁷Cl₂); 251, 28 M⁺ (³⁷Cl³⁵Cl); 249, 42 M⁺ (³⁵Cl₂); 162, 6 M⁺-C₃H₅NO₂; 145, 9, M⁺-C₃H₆NO₃; 133, 13; 88, 100, C₃H₆NO₂; 60, 56.

Microanalysis: Calculated for C₉H₉NO₃Cl₂, C 43.2%, H 3.6%, N 5.6%, Cl 28.4%; Found C 43.3%, H 3.8%, N 5.4%, Cl 28.8%.

### Preparation of DDHP (The Pirimicarb Metabolite)

A solution of 119 mg (0.5 mmol) of pirimicarb (Sigma-Aldrich) in 3 mL of methanol and 2.5 mL of 2 M aqueous sodium hydroxide was refluxed by heating in an oil bath at 120° C. for 1.5 hours. The reaction mixture was cooled in ice, 2 M aqueous hydrochloric acid added to adjust the pH from 14 to 6 and the solution lyophilized. The resulting solid was extracted 3 times with 10 mL of ethyl acetate. The combined extracts were evaporated to give 97 mg of white crystalline material which was purified by flash chromatography on a 120 mm×20 mm column of silica eluted with 3:2 ethyl acetate:isopropanol to give 57 mg of DDHP in 68% yield.

¹H NMR (CDCl₃): 1.88, s, 3H; 2.15, s, 3H, 3.12, s, 6H, 11.95, br, 1H.

¹³C NMR (CDCl₃): 10.0, 22.3, 37.3, 105.7, 152.0, 162.7, 165.7.

EIMS, m/z: 168, (M⁺+1), 167, M⁺, 166, (M⁺−1), 152, 138 (base peak), 124, 123, 110, 97, 83, 71, 69, 55, 53.

Characterisation and Identification of M. brisbanense JK1

MM (50 mL) supplemented with 10 mM glucose, 0.13 mM diuron and 18.7 mM ammonium chloride (as required) were inoculated with 0.2% of a homogeneous suspension of freshly grown M. brisbanense strain JK1 cells (0D₆₀₀=˜10). Cultures were incubated at 28° C. with shaking at 200 rpm, in the dark. Samples were taken at ˜24 hour intervals, and metabolites were extracted in 50% acetonitrile, filtered and analysed by HPLC.

Routine Gram staining and light microscopy were performed to examine bacterial morphology. The 16S rDNA gene of M. brisbanense strain JK1 was amplified with the universal primers 27f and 1492r (Lane, 1999), using colony-PCR with the high fidelity polymerase Pwo (Roche, Mannheim, Germany) and an Eppindorf Mastercycler® using a 55° C. annealing temperature and 72° C. elongation temperature. Amplicons were purified using the QIApick kit (Qiagen) and sequenced.

Construction and Screening of a pYUB415 Cosmid Library

Genomic DNA was extracted using a phenol:chloroform extraction method (Moore and Dowhan, 2002) from M. brisbanense strain JK1 and partially digested with Sau3AI to obtain fragments of approximately 40 kb. The fragments were purified after electrophoresis in a 0.8% low melt agarose (Scientifix) TAE gel and DNA was extracted by diluting and melting the agarose, which was removed after freezing by centrifugation, followed by ethanol-salt precipitation. The fragments were cloned into pYUB415, an E. coli-Mycobacterium shuttle vector (Bardarov and Jacobs Jr, unpublished) that had been digested with BamHI and dephosphorylated with SAP (Promega). The pYUB415 library was packaged into phage particles used to infect E. coli LE392MP cells, using MaxPlax Lambda packaging extracts (Epicentre), according to the manufacturers instructions.

Cosmid DNA was extracted from cultures of pooled (10 clones/pool) E. coli cosmid clones using QIAquick (Qiagen). The pooled cosmid DNA was transformed into M. smegmatis by electroporation and cells were grown on solid media. All growth on the plates was washed into McCartney bottles containing MM amended with 86 μM diuron and incubated at 37° C. for 2-10 days with monitoring for diuron degradation. M. smegmatis containing pYUB415 without an insert was used as a negative control. Cosmid 158 was digested using BamHI and BglII (Fermentas, NEB) and the fragments cloned into pYUB415, transformed into M. smegmatis and screened for diuron hydrolase activity.

Routine DNA sequencing was performed by Micromon DNA Sequencing Facility, Monash University, Melbourne and large DNA fragments (20-40 kb) were sequenced by the Australian Genome Research Facility (AGRF, University of Queensland, Brisbane) by shotgun sequencing with 8-fold sequence coverage. DNA was routinely visualised by electrophoresis in 0.5-1.5% agarose (Promega) gels in TAE buffer using ethidium bromide (Amresco).

### Expression Constructs and Conditions

PuhA and PuhB were amplified as N-hexa-histidine tagged fusions from pET14b (Novagen) in E. coli rosetta II cells (Novagen) and pMV261 (Stover et al., 1991) in M. smegmatis mc2. Protein without hexa-histidine tags was later expressed in M. smegmatis mc2. The PCR primers used in these cloning procedures are noted in Table 2 (SEQ ID NOs 5 to 13).

TABLE 2
Primers used for recombinant constructs, with
restriction sites underlined.

| | Restriction | |
|---|---|---|
| Primer name | site | Sequence |
| PuhA-petl4b fwd | NdeI | GGCCCATATGACCACCACCGCC |
| (SEQ ID NO: 5) | | ATCACCGACAT |
| PuhA-petl4b rev | BamHI | TGACGGATCCTCAGGCGGGATG |
| (SEQ ID NO: 6) | | CGCCGTGACGAG |
| PuhB-pET14b fwd | NdeI | GGCCCATATGAGCACCATCGCC |
| (SEQ ID NO: 7) | | ATCACCAATG |
| PuhB-pET14b rev | BamHI | TGACGGATCCTTACGCGGGGTG |
| (SEQ ID NO: 8) | | AGCAGTCACGAG |
| His AB pMV fwd | BamHI | TGACGGATCCAATGGGCAGCAG |
| (SEQ ID NO: 9) | | CCATCATCA |
| PuhA-pMV261 fwd | BamHI | GGCCGGATCCAATGACCACCAC |
| (SEQ ID NO: 10) | | CGCCATCACCGAC |
| PuhB-pMV261 fwd | BamHI | GGCCGGATCCAATGAGCACCAT |
| (SEQ ID NO: 11) | | CGCCATCACCAATG |
| PuhA pMV261 rev | HindIII | TGACAAGCTTTCAGGCGGGATG |
| (SEQ ID NO: 12) | | CGCCGTGACGAG |
| PuhB pMV261 rev | HindIII | TGACAAGCTTTTACGCGGGGTG |
| (SEQ ID NO: 13) | | AGCAGTCACGAG |

Pwo polymerase (Roche) and restriction enzymes NdeI, BamHI and HindIII (Fermentas) were used following manufacturers' protocols. Electro-competent E. coli strains JM109 (Promega) were utilised for transformation of ligation reactions (using Promega ligase). Protein expressions in E. coli were inoculated (1% v/v) with an overnight seed culture in LB and then incubated with shaking at 20° C. for 11 hours followed by induction with 0.1 mM isopropyl-β-D-thiogalactopyranoside (IPTG) for 3 hours. M. smegmatis expressions were amended with 0.05% tween 80 and grown in LB at 28° C. for 2-4 days with no induction. Clarified cell-free extracts were obtained by resuspending the bacteria in 25 mM Tris (pH 8) followed by 3 passes through a French pressure cell (Thermo) and centrifugation at 27,000 g (Beckman Avanti).

### Protein Quantification, Purification and Visualisation

Protein concentration was estimated using the Bio-rad protein assay (Bradford), with bovine serum albumin used as standard. Protein purity was monitored using sodium dodecyl sulphate-polyacrylamide gel electrophoresis (SDS-PAGE) stained with Coomassie brilliant blue. Where PuhA and B concentrations were estimated in semi purified extracts, SDS-PAGE gels were scanned using Labscan v5 (Amersham Biosciences) and band densitometry performed with Total lab TL100 (Nonlinear dynamics). Talon cobalt resin (BD Biosciences) was used for the purification of hexa-histidine tagged protein. Untagged protein was purified by ammonium sulfate (AS) precipitiation followed by hydrophobic interaction chromatography (HIC; Phenylsepharose), anion exchange chromatography (AEX; Macro Q) and size exclusion chromatography (SEC; Superose 6 or Sephadex 200). All columns were purchased from GE Healthcare. The hydrophobic interaction column was equilibrated with Tris (pH 8)/1 M ammonium sulfate and bound protein was then eluted with Tris (pH 8) over 400 mL. Protein was bound to the anion exchange column with Tris (pH 8) and eluted with a gradient of Tris (pH 8)/1 M NaCl over 500 mL. The buffer was exchanged to 25 mM HEPES/50 mM KCl (pH 8) during SEC and the protein was stable in this buffer at 4° C. for several weeks. The oligomeric structure was estimated with a Superose 6 size exclusion column calibrated with the following protein standards (kDa): thyroglobulin (669), ferritin (440), catalase (232) and aldolase (158). Standard tryptic peptide mass fingerprinting was used to confirm the identity of proteins excised from polyacrylamide gels by liquid chromatography electro spray ionisation mass spectroscopy/mass spectroscopy (LC ESI MS/MS) ion trap following the method of Campbell et al. (2008).

### Enzyme Assay Development

Metabolites from growth cultures and enzyme assays were analysed using HPLC or liquid chromatography mass spectrometry (LCMS). HPLC was performed using a Beckman Gold, with Gold Noveau software version for analysis of the data. A Phenomenex Aqua C18 (5μ, 125 Å, 250×4.60 mm) reverse phase column or Phenomenex Synergi reverse phase column with the same specifications was also used. The guard columns were Alltech Adsorbosil C18 (5μ, 7.5×4.6 mm) or Phenomenex C18 Octadecyl (4 mm L×3 mm ID). Generally analysis was performed using an isocratic method with a 1:1 mix of acetonitrile and water modified with 0.1% phosphoric acid. Flow rates ranged from 1-1.5 mL.min⁻¹ with the UV detector set at 250 nm. The LCMS system incorporated an Agilent 1100 series LC system with a time of flight detector fitted with an electrospray ionisation injector. Formic acid (0.1%) was used as modifier for LCMS. The mass analysis was generally done in positive ion mode with default settings, scanning 2 fragmentor voltages of 120 V and 225 V. Analysis of 3,4-dichorophenol and DDHP required negative ion mode.

A high throughput assay was developed to assist in kinetic characterisation of the proteins. This method relied on the formation of a coloured product with the reagent o-dianisidine bis(diazotized) zinc double salt (Fast Blue B Salt; FBBS; Sigma). The method is based on that reported by Van Asperen (1962) for the detection of α-napthol (Aₘₐₓ=600 nm). In the current case, the diazo product formed with the aniline metabolites of the phenylureas to produce yellow colours with Absₘₐₓ˜450 nm, although the magnitude of the absorbance and hence sensitivity of the assay varied between anilines. The FBBS (9 mg) was dissolved in MilliQ (MQ) water (9.75 mL) followed by addition of 5.25 mL of 10% SDS. A 1/6 addition was made to an enzyme assay and the addition of the reagent stopped the reaction. A pipette tip dipped in butanol was used to remove any bubbles prior to reading A₄₅₀ nm on a Spectra MAX190 spectrophotometer (Molecular Devices). All enzyme assays were performed in 25 mM MOPS, 50 mM KCl (pH 6.9), containing 0.4% acetonitrile at 25° C. unless otherwise specified and corrected for any non-enzymatic hydrolysis or background absorbance. P-nitrophenol produced by the hydrolysis of paraoxon and N-dimethyl, O-4-nitrophenyl carbamate (DMNPC) was measured at A₄₀₀ nm while kinetic assays with the linuron ester and pirimicarb were analysed by LCMS.

### Enzyme Characterisation

The influence of solvents (acetonitrile and methanol) was examined by using 7 concentrations from 0.4-18.2% over 1 hour duplicate assays with 77 μM diuron and monitored by LCMS. The effect of temperature on the activity of the enzymes was determined in two ways: firstly, the temperature at which reaction was catalysed was controlled at 12 temperatures ranging from 2-60° C. with 154 μM diuron; secondly, enzyme was preincubated at 6 temperatures from 30-55° C. for 10 minutes prior to 1 hour assays at 25° C. The residual activity data was obtained by LCMS and fitted to the Boltzmann function (eq 1)

$$y = {y}^{min} + \frac{( {y}^{max} - {y}^{min} )}{1 + {e}^{\frac{( T - {T}_{m} )}{λ}}} \qquad ( 1 )$$

where the activity (y) is predicted by y^{min} and y^{max}, the lower and upper asymptotes, T, the temperature of pre-incubation, Tₘ, the melting temperature at Δy/2, and λ, a parameter that describes the shape of the curve between the asymptotes. To monitor the effects of pH a constant ionic strength buffer was prepared with 50 mM acetic acid, 50 mM MES and 100 mM Tris (Ellis and Morrison, 1982) at 10 pH values from 5-9.5. Duplicate kinetic curves were obtained at each pH value. All assays were corrected for any non enzymatic hydrolysis occurring using negative controls. Standards were injected 3 times each and used to construct a quantitation curve (linear) of 3,4-dichloroaniline (DCA). Data for both enzymes were fitted to either the Michaelis-Menten equation or to the following equation for substrate inhibition: ν=Vₘₐₓ/(1+I/Kᵢₛ+Kₘ/S). In some instances the solubility limit of the compound prevented any velocity measurement above the Kₘ; where a regression could not be performed the slope of v/[S] or the k_{cat}/Kₘ was recorded. A bell shaped model (eq 2)

$${( y )}^{H} = \frac{{( y )}^{max}}{( \frac{{10}^{- pH}}{{10}^{- {pK}_{a} 1}} ) + ( \frac{{10}^{- {pK}_{a} 2}}{{10}^{- pH}} ) + 1} \qquad ( 2 )$$

was used to fit an activity y, either log(k_{cat}) and log(k_{cat}/Kₘ) vs. pH. (y)^{max} is the plateau value and pKₐ¹ and pKₐ² are the apparent pKₐ values for the acidic and basic groups, respectively. Equations were fitted to the data using Kaleidagraph v3.6 (Synergy Software). The substrate pKₐ values were obtained via an online database www.syrres.com/esc/physdemo.htm (Syracuse Research Corporation).

### Genomic and Phylogenetic Analysis

FGENESB (http://www.softberry.com) was used to visualise open reading frames (ORFs) and manually confirmed. Nucleotide and translated amino acid sequences were analysed using the Basic Local Alignment Search Tools (BLAST; Altschul et al., 1997; and Schaffer et al., 2001). Identification of promoters utilised the program BPROM (www.softberry.com). BioEdit v7.0.5.2 (Hall, 1999) was used to identify restriction endonuclease recognition sites, analyse the promoter regions and calculate nucleotide identity scores.

Analysis of the PuhB amino acid sequence was performed with the NCBI conserved domain search, which displayed 33 sequences most dissimilar from the query (as determined by BLAST) and shared similar domain architecture. After removing one sequence that did not align well by ClustalW (gi|28926800), a phylogeny was constructed. The complete deletion option was applied with the Dayhoff (PAM) scoring matrix and neighbour joining trees were constructed using Molecular Evolutionary Genetics Analysis (MEGA) software v4.0 (Tamura et al., 2007), which were bottstrapped 1000 times.

To find other sequences with NxH motifs the search tools BLASTp and tBLASTn were applied to the NCBI databases; nr, patent, Env samples (protein), nr/nt, GSS, WGSS, HTGS, env samples (DNA) and genomic reference sequences as at Mar. 1, 2008.

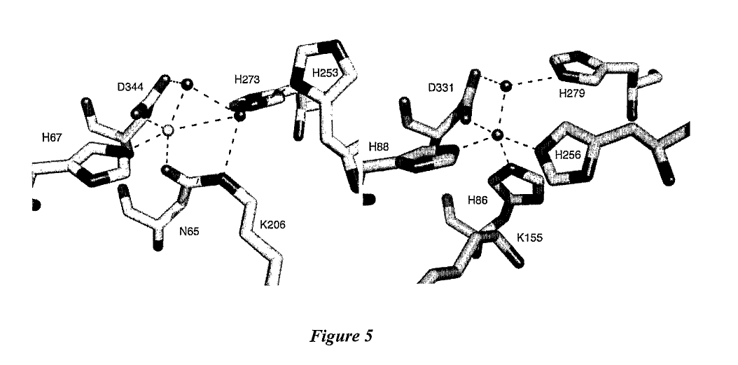

A homology model of the active site of PuhB was made, using an alignment with the closest structural homolog, 2QS8 (FIG. 4). Conserved metal ligands and second-shell residues were threaded onto the structure of 2QS8 using the program Swiss-PDB Viewer (Kaplan and Littlejohn, 2001), and the single difference in the active site, the substitutuion of H65 by N65, was manually adjusted using the program COOT (Emsley and Cowtan, 2004).

### Metal Analysis

Purified enzyme was concentrated with a 100 kDa Amicon spin column (Millipore) and quantified by measuring A₂₈₀ with a Nanodrop spectrophotometer (Nanodrop Technologies). The values were adjusted for the theoretical A₂₈₀ extinction co-efficient, 1A₂₈₀=1.03 mg.mL⁻¹ for PuhA and 1A₂₈₀=0.98 mg.mL⁻¹ for PuhB. These samples were analysed using an in-house method (NATA accredited) for food samples by the National Measurement Institute. Briefly 0.2 mL of sample was digested in 2 mL concentrated nitric acid in a polypropylene tube by heating on a steam bath for 2 hours, then diluted to 30 mL with high purity water and analysed by induction coupled plasma-atomic emission spectroscopy (ICP-AES) and induction coupled plasma-mass spectroscopy (ICP-MS).

### Example 2

### Isolation of a Diuron Degrading Bacterium

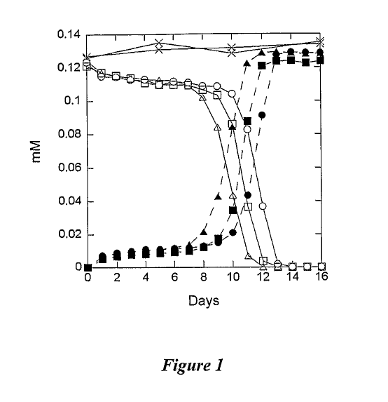

Enrichment cultures in which diuron was the sole nitrogen source were set up from four diuron-exposed soil samples. Diuron was mineralised by one culture, as detected by HPLC, and this culture was serially subcultured 8 times without loss of activity. A pure strain of Gram-positive bacteria with diuron hydrolase activity was isolated from the fourth subculture by dilution plating. The 16S rDNA was sequenced and found to have 99.7% identity to M. brisbanense ATCC 49938T (AY012577), a member of the M. fortuitum third biovariant complex of fast growing Mycobacteria (Schinsky et al., 2004). The isolate was thus designated M. brisbanese JK1 and its degradation of diuron was confirmed by monitoring the loss of diuron and accumulation of the metabolite DCA in liquid culture with diuron in the presence of an alternate source of nitrogen (FIG. 1).

### Example 3

### Cloning of PuhB

A cosmid library was prepared from the total DNA of M. brisbanense JK1 using the E. coli-Mycobacterium shuttle vector pYUB415. As no diuron hydrolysis was detected in any of the 600 cosmid clones screened in E. coli LE 392 MP (data not shown), 320 cosmid clones (expressed in pools of 10) were screened in M. smegmatis mc2. A single pool was identified with diuron hydrolase activity. A single cosmid (158) was found that conferred diuron hydrolase activity on M. smegmatis mc2 when the cosmids from this pool were screened individually.

Using restriction digests, the ca. 40 kb insert was reduced to an active 15 kb BglII fragment, then an active 2.5 kb BamHI fragment. The 2.5 kb BamHI fragment was sequenced by primer walking to reveal one complete ORF. A 1386 bp ORF was found to have 79% nucleotide identity to the known diuron hydrolase encoding gene puhA, and the encoded protein is 82% identical to PuhA. The newly identified gene was named puhB.

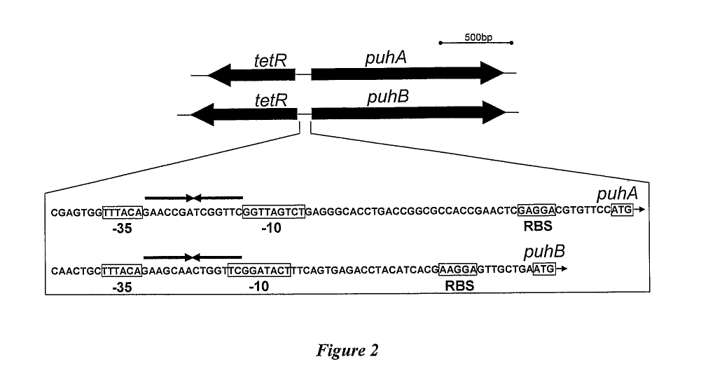

The plasmid containing puhA (pHRIM622; (Turnbull et al., 2001b)) and the cosmid containing puhB were sequenced and compared. Similarities in the promoter regions at −10, −35, ribosome binding site (RBS) and palindromic sequences were identified (FIG. 2). Syntenous tetR regulatory genes were identified (78% amino acid identity) in both sequences, although there was no further identity. The presence of conserved regulatory genes and a potential transcription factor binding site (palindromic sequence) in the promoter region suggests that these hydrolase genes may be located adjacent to the genes for their regulatory proteins.

### Example 4

### Sequence Analysis

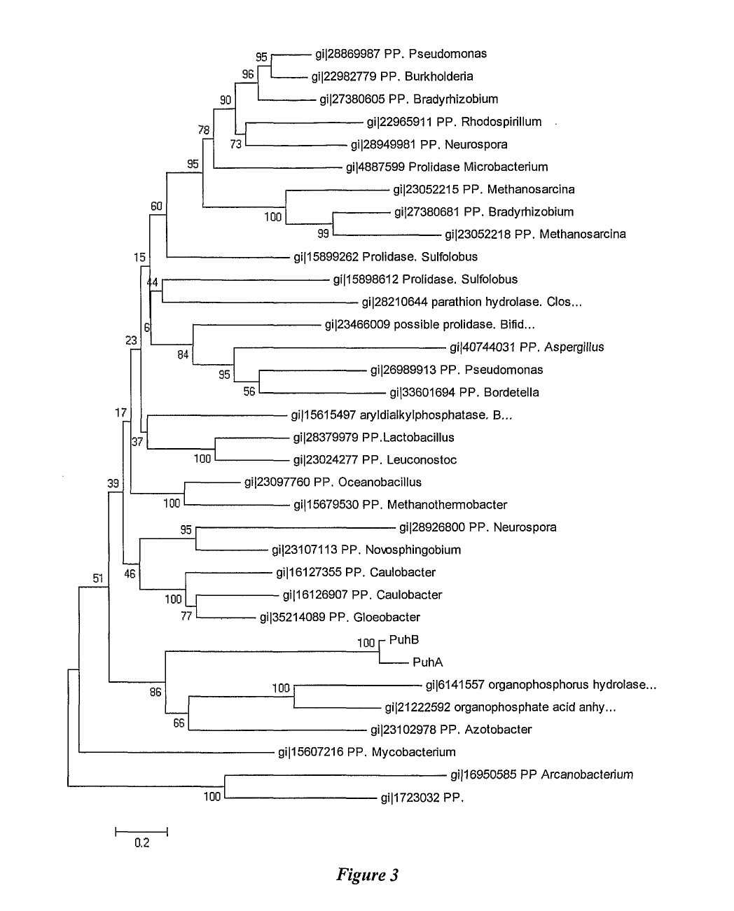

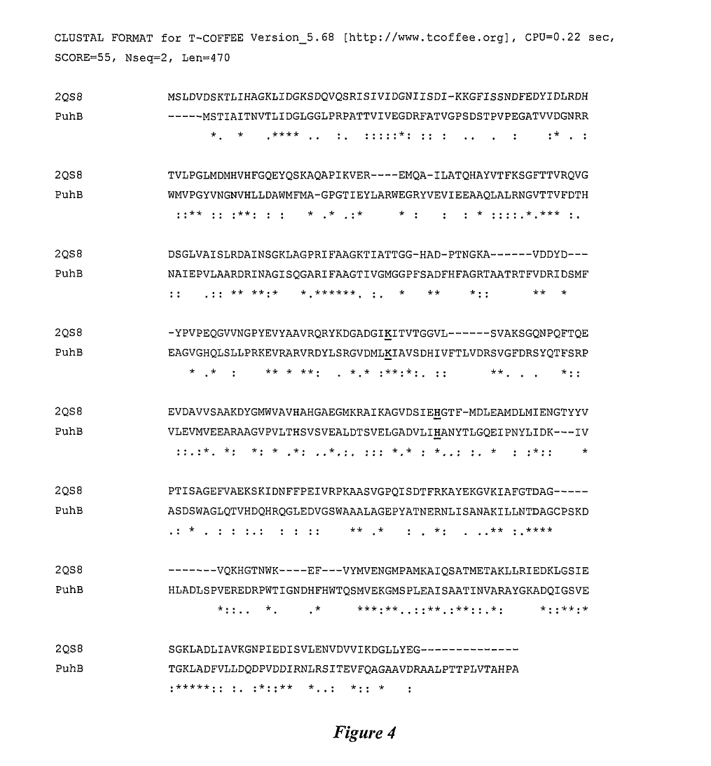

An NCBI conserved domain search revealed that PuhA and B cluster within the metal dependant hydrolase A (CD01299) sub-family, which belongs to the amidohydrolase superfamily. A neighbour joining phylogeny was constructed using the 32 most diverse sequences (FIG. 3). The closest branch (with 86% bootstrap support) to the PuhA and B sequences contained the organophosphorus acid hydrolase from Arthrobacter sp. strain B-5 (Ohshiro et al., 1999). Generally, an HxH motif on strand 1 of amidohydrolase proteins is conserved, and is thought to be essential for metal binding (Seibert and Raushel, 2005). However, the phenylurea hydrolases have a variant NxH motif in this position. Indeed, when BLAST was used to align PuhB against the entire non-redundant database (NCBI), only four short DNA sequences were identified that shared the NxH motif, confirming its rarity.

A comprehensive review by Seibert and Raushel (2005), classified the amidohydrolase superfamily into 7 subtypes on the basis of their metal binding ligands. The closest relatives of the phenylurea hydrolases with known structures are 2QS8, an Xaa-Pro Dipeptidase from Alteromonas macleodii, 2P9B, a putative prolidase from Bifidobacterium Longum, and 2GOK, an imidazolonepropionase from Agrobacterium tumefaciens, all of which belong to subtype III. This subtype contains a mononuclear active site containing either Zn or Fe co-ordinated by an HxH motif from β-strand 1, an H from β-strand 5 and a D from β-strand 8. A conserved H on β-strand 6 is located in the second shell of the active site and thought to be hydrogen bonded to either the catalytic water molecule or the substrate. Interestingly, these structures also have a lysine residue in the active site, although it is not coordinated by the metal ion. In PuhA and B, the first H of the HxH motif is replaced by N, suggesting that this asparagine is involved in metal co-ordination. Asparagines are known to be involved in metal ion coordination in other metalloenzymes (Jackson et al., 2007). A crystal structure will be necessary to confirm the active site architecture but, the substitution of a histidine metal ligand in the PUHs with asparagine differentiates them from other amidohydrolase subgroups and they thus form a novel structural subtype (VIII; Table 3).

A homology model of a part of the active site of the phenylurea hydrolases, based on the structure of 2QS8, is shown in FIG. 5. The only residue in the 2QS8 active site not conserved in the phenylurea hydrolases is the first histidine, which is replaced by an asparagine in the model. When compared with the active site structure of 200K, the largest difference is the position of the histidine metal ligand from β-strand 5; in 2QS8 it does not coordinate the metal ion, and is replaced by a water molecule, whereas in 2GOK it does coordinate to the active site metal ion. It is unclear whether this is functionally significant or is a crystallization artefact. Other than this, the structures both contain histidine and lysine residues in the active site that are not metal ligands, although the lysines are not in analogous positions. Finally, a water molecule, tightly coordinated to the active site metal ion is present. The catalytic roles of the metal-bound water and second-shell histidine and lysine residues in the phenylurea hydrolases is discussed below.

TABLE 3
Amidohydrolase subtypes I-VIII, modified from
Seibert and Raushel (2005), with the phenylurea
hydrolases forming the new subtype VIII.

| | | | strand | | | | | | | |
|---|---|---|---|---|---|---|---|---|---|---|
| subtype | metals | positions | 1 | 2 | 3 | 4 | 5 | 6 | 7 | 8 |
| I | Zn, Ni | α, β | HxH | | | K | H | H | | D |
| II | Zn | α, β | HxH | | | E | H | H | | D |
| III | Zn, Fe | α | HxH | | | | H | h^{b} | | D |
| IV | Fe | β | hxh^{a} | | E | | H | H | | d^{b} |
| V | Zn | β | hxh^{a} | C | | | H | H | | d^{b} |
| VI | Zn | α, β | HxD | | E | | H | H | | d^{b} |
| VII | | | HxH | | | | H | | | d^{b} |
| VIII | Zn | α | NxH | | | | H | H | | D |

^{a}Present in the active site, these residues do not appear to ligate a metal or in some cases can accommodate a second metal ion that is not required for catalysis.
^{b}Hydrogen bonds link these residues to the hydrolytic water molecule

### Example 5

### Expression and Purification

Initially, PuhA and B were natively expressed in and purified from A. globiformis D47 and M brisbanense JK1, respectively. The constitutively expressed enzymes were purified by first obtaining the soluble fraction of a 30-50% AS fractionation, followed by HIC, and finally by SEC. A purification factor of 51 was achieved for PuhA (Table 4). The identity of a ˜50 kDa band on SDS-PAGE consistent with the molecular weight of PuhA was confirmed by tryptic digest peptide fingerprinting, which gave 58% sequence coverage including the predicted N and C termini. The N terminal fragment was missing the initial methionine, consistent with common bacterial exopeptidase processing (Giglione et al., 2004). Unfortunately, native PuhB lost activity during purification. However, by monitoring activity using DMNPC, the native molecular weights for PuhA and PuhB were both calculated to be ˜310 kDa using calibrated SEC, consistent with a 300 kDa hexamer comprised of six ˜50 kDa subunits. The predicted monomer masses (excluding the N terminal methionine) are 48.752 and 49.546 kDa for PuhA and PuhB, respectively.

TABLE 4
Purification of native PuhA and PuhB.

| | CFE | AS | HIC | SEC |
|---|---|---|---|---|
| Native PuhA | | | | |
| Activity | 0.05 | 0.18 | 0.73 | 2.79 |
| (nmol DCA/μg/hour) | | | | |
| Purification factor | — | 3.28 | 13.37 | 51.19 |
| Estimated specific | — | — | 10.20 | 7.02 |
| activity (mol/mol/min) | | | | |
| Native PuhB | | | | |
| Activity | 0.05 | 0.24 | 0.24 | 0.04 |
| (nmol DCA/μg/hour) | | | | |
| Purification factor | — | 4.8 | 4.8 | 0.8 |

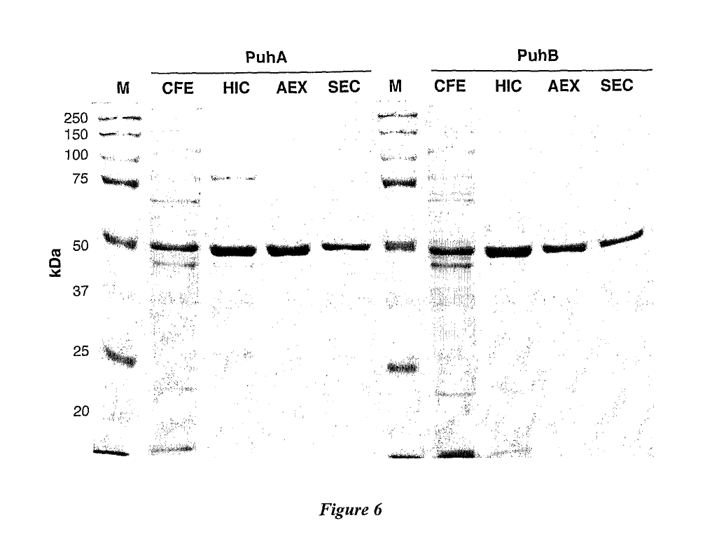

For recombinant expression, PuhA and B were cloned into the pET14b expression vector and expressed in E. coli Rosetta 2 DE3 cells at 20° C. for 11 hours followed by induction with 0.1 mM IPTG for 3 hours. Over-expressed bands were observed at the correct molecular weights as judged by SDS-PAGE. However, the specific activity in the soluble fraction was ˜10 fold lower for PuhA compared to the native expression, and no activity was detected for PuhB (data not shown). The proteins were then cloned into the Mycobacterium expression vector (pMV261) and expressed in M. smegmatis. In this instance, the specific activity in the soluble fraction of cells expressing PuhA was comparable to that measured in the native expressions, and the soluble fraction of cells expressing PuhB also displayed diuron hydrolase activity. Both proteins were then purified to homogeneity by AS precipitation, HIC, AEX, and SEC (FIG. 6; Table 5). The purified recombinant proteins were catalytically active, of the predicted monomeric molecular weight as judged by SDS-PAGE, and were judged by SEC to oligomerise into ca. ˜300 kDa complexes, most likely hexamers.

TABLE 5
Purification of PuhA and PuhB expressed in M. smegmatis.

| | PuhA | | | | PuhB | | | |
|---|---|---|---|---|---|---|---|---|
| | CFE | HIC | AEX | SEC | CFE | HIC | AEX | SEC |
| Total protein | 1427.5 | 85.1 | 27.4 | 15.8 | 1856.8 | 90.0 | 31.9 | 24.9 |
| (mg) | | | | | | | | |
| Activity (mM | 0.3 | 4.3 | 6.2 | 7.8 | 0.1 | 1.4 | 3.9 | 4.3 |
| PNP/mg/min) | | | | | | | | |
| Purification | | 17 | 25 | 31 | | 15 | 41 | 45 |
| factor | | | | | | | | |

### Tryptic Digestion

Bands from SDS-PAGE were excised and diced into ˜1 mm cubes. These were washed 2-3 times in 50 μL 1:1 acetonitrile/25 mM ammonium bicarbonate to remove coomassie stain, followed by a wash in 100% acetonitrile. The sample was vacuum dessicated then rehydrated in a 1/30 dilution of Promega sequencing grade trypsin solution (V5113) in 25 mM ammonium bicarbonate (pH 7.8). Dessication was acheived by incubation at 37° C. overnight. The sample was rehydrated in 0.1% Formic acid and filtered through 0.22 μm membrane (Millipore) for analysis by MS-TRAP. Analysis was kindly performed by P. Campbell utilising an Agilent 1100 liquid chromatography system fitted with Zorbax SB-C18 (5 μm, 150×0.5 mm) column combined with an Agilent XCT ion trap mass spectrometer using an electrospray ion source with micronebuliser as described in Campbell et al., 2008. Tryptic digest data was analysed using Spectrum Mill MS Proteomics Workbench Rev A.03.02.060a Agilent Technologies using the default settings. The data was first auto validated against a database of contaminants including trypsin, keratin and acrylamide, then screened against PuhA and finally using the NCBI Eubacteria nr protein database (11.06). The reverse database was included to exclude spurious matches of large peptides whose fragmentation appeared to match the forward database as well as the reverse.

### Example 6

### Metal Analysis

Sequence analysis places PuhA and B within the metal dependent amidohydrolase superfamily. Thus, the present inventors determined the identity of the active site metal in these proteins through inductively coupled plasma-atomic emission spectrometry (ICP-AES) and inductively coupled plasma-atomic mass spectrometry (ICP-MS). This analysis showed that, of the second row transition metals tested (Co, Cu, Fe, Mn, Ni, and Zn), Zn was the most abundant, with a calculated stochiometry of 0.6 Zn ions per active site in both PuhA and PuhB. Presumably, there was a proportion of apo-enzyme owing to over expression, in addition to promiscuous incorporation of other metal ions at lower levels (˜5-10%). This result strongly suggests that the phenylurea hydrolases are mononuclear zinc enzymes.

### Example 7

### Enzyme Stability

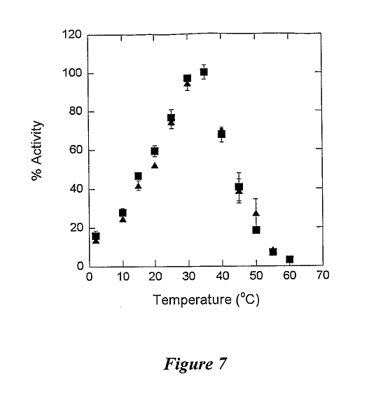

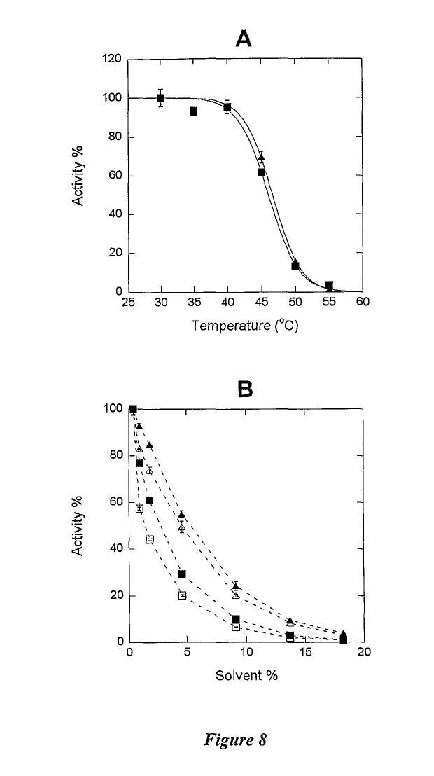

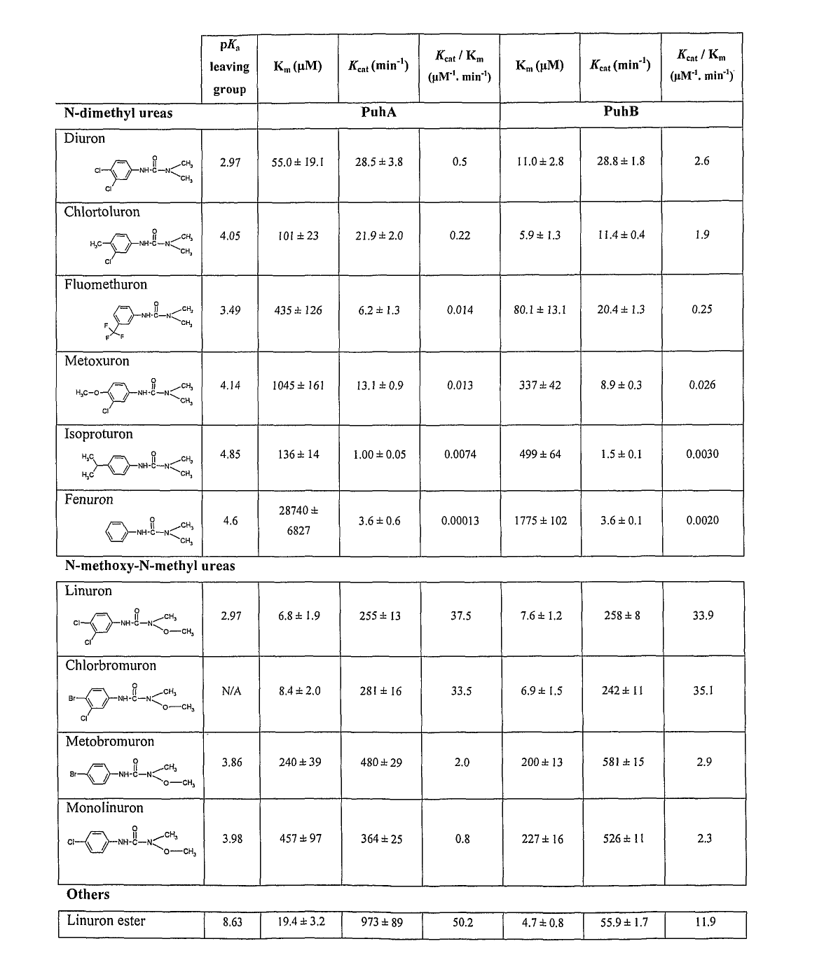

Thermophilicity of the enzymes was tested by determining the activity of PuhA and PuhB against diuron at temperature between 2-35° C. (FIG. 7). Activity maxima for both enzymes were between 30 and 35° C. The thermal stability of both enzymes was then tested using measurement of residual activity after incubation at a number of temperatures. Again, both enzymes behaved almost identically, retaining significant activity after 10 minutes incubation at 40° C., beyond which they began losing activity. These results were plotted and a Boltzmann equation of decay was fitted, Tₘ indicating 50% activity retained at 46.6° C. for PuhA and at 46.0° C. for PuhB (FIG. 8A). Finally, stability in the presence of the solvents acetonitrile and methanol (within a range of 1-18% v/v) was determined. PuhB appeared to have a slightly increased resilience to the use of these solvents compared with PuhA, although both enzymes were significantly inhibited in the presence of both methanol and acetonitrile (FIG. 8B).

### Example 8

### Substrate Range and Kinetic Properties

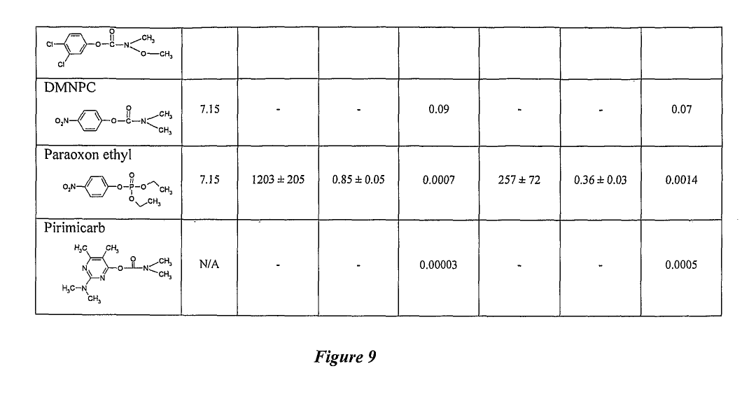

The turnover of fifteen phenylurea herbicides by both enzymes was monitored using LCMS (FIG. 9). There was no detectable hydrolysis of 5 phenylureas (dimefuron, neburon, siduron, tebuthiuron, and thidiazuron), which are significantly bulkier than the other 10 substrates, suggesting a possible basis for their resistance to catalysis. The 10 remaining phenylurea substrates can be grouped by the identities of their side chains as either N-dimethyl or N-methoxy-N-methyl compounds, were turned over with rates (k_{cat}) between 1 and 526 min⁻¹. The turnover rate (k_{cat}) of the N-methoxy-N-methyl substrate linuron was ca. ˜9 fold higher than that of the N-dimethyl substrate diuron, which is otherwise structurally identical. In fact, despite the isolation of both PuhA and B from bacteria selected for diuron degradation, both enzymes displayed the greatest catalytic efficiency with linuron (k_{cat}/Kₘ values of 37.5 and 33.9 μM⁼¹.min⁼¹, respectively). Both enzymes had significantly higher k_{cat} values for the other N-methoxy-N-methyl substrates as well. The greater electron withdrawing character of the oxygen substituent in the N-methoxy-N-methyl substrates may contribute to the faster turnover rates observed for these compounds.

PuhA catalyses the turnover of the N-methoxy-N-methyl phenylurea linuron more efficiently at low concentrations than the N-dimethyl phenylurea diuron (Kₘ 6.8 μM vs. 55.0 μM). In contrast, PuhB has comparable Kₘ values for the catalysis linuron and diuron hydrolysis (7.6 μM vs. 11.0 μM). Indeed, PuhB displays globally lower Kₘ values for the N-dimethyl substrates than PuhA, with the exception of isoproturon. Thus, it is likely that some of the sequence differences between the PuhA and B sequences result in structural differences in the substrate binding pocket, which have made PuhB a more efficient catalyst of N-dimethyl phenylurea hydrolysis. Futhermore, the substitutions of the N′-phenyl groups of the phenylurea substrates also appear to significantly affect the concentration at which both enzymes can efficiently catalyse the reaction (Kₘ).

The kₙₒₙ value of diuron in neutral buffer was estimated from the work of Salvestrini et al. (2002) at 2.8×10⁻¹⁰ sec⁻¹. Thus PuhB with a k_{cat} for diuron of 0.48 sec⁻¹ offers a rate enhancement k_{cat}/kₙₒₙ of 1.7×10⁹. When compared with the related mononuclear zinc or iron subtype III enzymes, adenosine deaminase (ADA) and cytosine deaminase (CDA). The ADA k_{cat} of 370 sec⁻¹ with a 2.1×10¹² rate enhancement and CDA k_{cat} of 300 sec⁻¹ with 1.1×10¹² rate enhancement are far superior (Frick et al., 1987; Snider et al., 2000).

In addition to the turnover of the phenylureas, PuhA and PuhB both displayed significant promiscuous activity, catalysing the hydrolysis of carbamate and organophosphate compounds (FIG. 9). The turnover rates for paraoxon ethyl were only slightly lower than isoproturon, the poorest phenylurea substrate however pirimicarb was the poorest substrate examined for both enzymes with 10⁴ and 10³ fold lower catalytic efficiencies than diuron for PuhA and PuhB, respectively. A carbamate analogue of linuron was synthesised (in which the amide bond to the leaving group was replaced by an ester bond) to compare the hydrolysis of ester and amide bonds by the enzymes in otherwise similar substrates. Purified PuhA and PuhB were both found to hydrolyse this substrate as efficiently as the phenylurea (FIG. 9), suggesting that these enzymes are general hydrolases, and that their catalytic specificity for the hydrolysis of an amide bond (as opposed to an ester or phosphoester bond) in phenylureas is a consequence of the shape of the substrate binding pocket.

Previously, a native soluble extract of A. globiformis D47 was shown to hydrolyse the carbamate DMNPC (Robinson, 2003), which was confirmed using purified enzyme in this study (FIG. 9). DMNPC was turned over at a higher efficiency than poor phenylurea substrates, such as metoxuron. The carbamate pirimicarb was also slowly turned over by PuhA and B, although neither enzyme catalysed the hydrolysis of two other carbamates that were also tested (carbaryl and thiobencarb). Interestingly, the organophosphate ethyl paraoxon was also turned over by both proteins, while the phosphothionate methyl parathion was not. This is interesting in light of the sequence homology between the phenylurea hydrolases and the organophosphorus acid hydrolase from Arthrobacter sp. strain B-5 (Ohshiro et al., 1999).

### Example 9

### Catalytic Mechanism

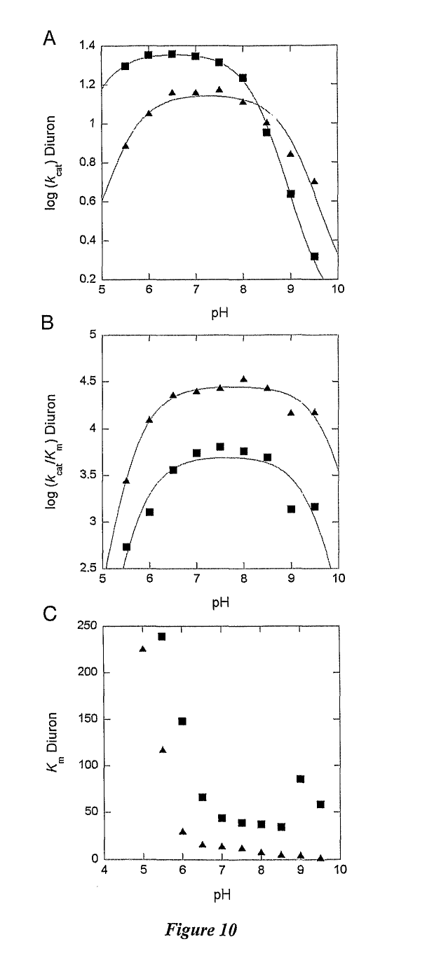

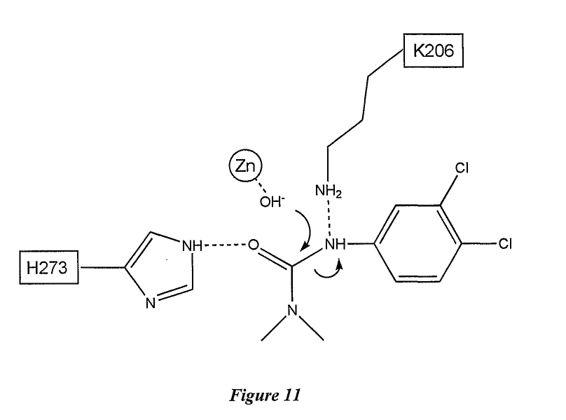

pH-activity analysis was performed to probe the mechanism of phenylurea hydrolysis by the phenylurea hydrolases. As seen in FIG. 10, both enzymes display broad pH optima for k_{cat}/Kₘ and k_{cat} between 6.5 and 8.5. The plots of log k_{cat} and log k_{cat}/Kₘ provide some interesting mechanistic information about PuhA and B. Firstly, there is a significant increase in the catalytic rate between pH 4 and 6 in both enzymes, which is likely to be a result of the generation of a nucleophilic hydroxide by the active site metal ion, in concert with D344 and possibly H273 (PuhB numbering). There is also a large increase in the Kₘ value below pH 6. This is consistent with the histidine residue (H273) in the second shell of the active site participating in substrate binding/coordination. There is a decrease in catalytic efficiency with a pKₑ of ˜10 in both enzymes (Table 6), which can be attributed to the deprotonation of the lysine (K206) residue in the vicinity of the active site. A plausible role for the amine group of a lysine in the active site of a hydrolase would be in stabilization of the developing negative charge on the leaving group. Based on these data, a catalytic mechanism is proposed in FIG. 11. Finally, both k_{cat} and k_{cat}/Kₘ are affected by pH, suggesting hydrolysis of the N—C bond is rate limiting. PuhA shows a small increase in Kₘ at basic pH that is not seen in PuhB, suggesting the substrate binding pockets of these enzymes may not be identical.

TABLE 6
The acidic and basic shoulders.

| Enzyme | pKₑ1 | pKₑ2 | pKₑₛ1 | pKₑₛ2 |
|---|---|---|---|---|
| PuhA | 4.2 | 8.9 | 5.1 | 10.2 |
| PuhB | 5.0 | 9.6 | 5.0 | 10.6 |

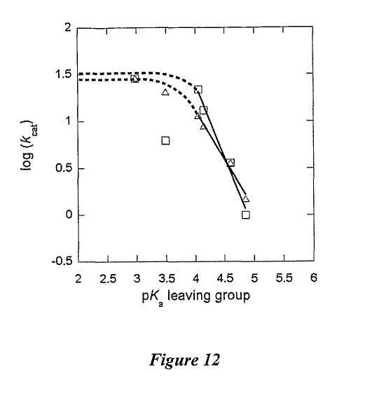

To investigate the kinetic effect of the N′-phenyl groups on the turnover rate, Bronsted plots (pKₐ phenyl group/log k_{cat}) were made for the turnover of the N-dimethyl substrates (FIG. 12). The aim of this experiment was to quantify the effect of the electronic properties of the leaving group on the catalytic rate. Although the range is relatively small (pKₐ values between 3-5), this is the largest range possible for the activity spectrum of the enzymes. The relationship between log k_{cat} and pKₐ is non-linear: the slope between pKₐ 4-5 is negative, giving β_{lg} values of −1.6 for PuhA and −1.1 for PuhB, indicating a large degree of bond cleavage in the transition state. Linear functions were fitted to this portion of the data (r² 0.98 for both enzymes), while the curve appears to flatten at pH values below 4. A change in the slope of the leaving group dependence is usually attributed to a change in catalytic mechanism or rate-limiting step (Hong and Raushel, 1996; McCain et al., 2002). This suggests that for highly electron withdrawing leaving groups with pKₐ values below 4, cleavage of the N—C bond may no longer be the rate limiting step.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

All publications discussed and/or referenced herein are incorporated herein in their entirety.

The present application claims priority from U.S. 61/134,442 filed 9 Jul. 2008, the entire contents of which are incorporated herein by reference.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

### References

Abdullah et al. (1986) Biotechnology 4:1087,
Altschul et al. (1997) Nucleic Acids Res. 25:3389-402,
Bardarov and Jacobs Jr. Unpublished data,
Canna-Michaelidou and Nicolaou. (1996) Sci. Total Environ. 193:27-35,
Campbell et al. (2008) Insect Biochem. Mol. Biol. 38:950-958,
Capecchi (1980) Cell 22:479-488,
Cheng et al. (1996) Plant Cell Rep. 15:653-657,
Clapp (1993) Clin. Perinatol. 20:155-168,
Cullington and Walker (1999) Soil Biology and Biochemistry 31:677-686,
Curiel et al. (1992) Hum. Gen. Then 3:147-154,
Eglitis et al. (1988) Biotechniques 6:608-614,
El-Deeb et al. (2000) Folia Microbiol. (Praha) 45:211-6,
Ellis and Morrison (1982) Methods Enzymol. 87:405-426,
Emsley and Cowtan (2004) Acta Crystallogr. D. Biol. Crystallogr 60:2126-32,
Engelhardt et al. (1971) Appl. Microbiol. 22:284-8,
Engelhardt et al. (1973) Appl. Microbiol. 26:709-18,
Field et al. (2003) J. Environ. Qual. 32:171-9,
Frick et al. (1987) Bioorg. Chem. 15:100-8,
Fujimura et al. (1985) Plant Tissue Culture Letters 2:74,
Gerecke et al. (2001a) Environ. Sci. Technol. 35:3915-23,
Gerecke et al. (2001b) J. Chromatogr. A 930:9-19,
Giacomazzi and Cochet (2004) Chemosphere 56:1021-32,
Giglione et al. (2004) Cell Mol. Life Sci. 61:1455-74,
Gooddy et al. (2002) Sci. Total Environ. 297:67-83,
Graham et al. (1973) Virology 54:536-539,
Grant et al. (1995) Plant Cell Rep. 15:254-258,
Gordon et al. (1999). Chemical-Biological Interactions 14:463-470,
Hall (1999) Nucl. Acids. Symp. Ser. 41:95-98,
Harayama (1998), Trends Biotechnol., 16: 76-82,
Hong and Raushel (1996) Biochem. 35:10904-12,
Jackson et al. (2007) J. Mol. Biol. 367:1047-62,
Jacobs et al. (1991) Methods Enzymol. 204:537-555,
Kaplan and Littlejohn (2001) Brief Bioinform. 2:195-7,
Koziel et al. (1996) Plant Mol. Biol. 32:393-405,
Lane (1999) Nucleic Acid Techniques in Bacterial Systematics (Stackebrandt and Goodfellow, Eds.), John Wiley & Sons, Inc., pp. 115-148,
LeJuene et al. (1998). Nature 395:27-28,
Lu et al. (1993) J. Exp. Med. 178:2089-2096,
Ohshiro et al. (1999) J. Biosci. Bioeng. 87:531-4,
Okamura (2002) Chemosphere 48:43-50,
Osano et al. (2002) Environ. Pollut.119:195-202,
McCain et al. (2002) J. Biol. Chem. 277:11190-200,
Moore and Dowhan (2002) Curr. Protoc. Mol. Biol. (Ausubel et al. Eds), Greene Publishing Associates and Wiley-Interscience, Unit 2.1A,
Needleman and Wunsch (1970) J. Mol. Biol. 48:443-453,
Petrikovics et al. (2000a). Toxicology Science 57: 16-21,
Petrikovics et al. (2000b). Drug Delivery 7: 83-89,
Salvestrini et al. (2002) Chemosphere 48:69-73,
Sambrook et al. (1989) Molecular cloning: a laboratory manual, Cold Spring Harbor Laboratory Press, Cold Spring Harbor, N.Y.,
Schaffer et al. (2001) Nucleic Acids Res. 29: 2994-3005,
Schinsky et al. (2004) Int. J. Syst. Evol. Microbiol. 54:1653-67,
Seibert and Raushel (2005) Biochem. 44:6383-91,
Sinning (1992) Trends Biochem. Sci. 17:150-4,
Snider et al. (2000) Biochem. 39:9746-53,
Sorensen and Aamand (2003) Pest. Manag. Sci. 59:1118-24,
Sorensen (2003) FEMS Microbiol. Ecol. 45:1-11,
Sorensen et al. (2008) Appl. Environ. Microbiol. 74:2332-40,
Spliid and Koppen (1998) Chemosphere 37:1307-16,
Stein et al. (1984) J. Cell Biochem. 24:243-59,
Stover et al. (1991) Nature 351:456-60,
Robinson (2003) BSc project, Department of Chemistry, University of Warwick,
Tamura et al. (2007) Mol. Biol. Evol. 24:1596-9,
Thurman et al. (2000) Sci. Total Environ. 248:189-200,
Tixier et al. (2002) Chemosphere 46:519-26,
Toriyama et al. (1986) Theor. Appl. Genet. 205:34,
Turnbull et al. (2001a) Biol. and Fertility of Soils 33:472-476,
Turnbull et al. (2001b) Appl. Environ. Microbiol. 67:2270-5,
Van Asperen (1962) J. Insect Physiol. 8:401-414,
Wallnofer (1969) Weed Res. 9:333-339,
Wagner et al. (1992) Proc. Natl. Acad. Sci. USA 89:6099-6103,
Wauchope et al. (1992) Rev. Environ .Contam. Toxicol. 123:1-155,
Wessels and Van der Veen (1956) Biochem. Biophys. Acta 19:548-9,
Widehem et al. (2002) Chemosphere 46:527-34,
Wustner et al. (1978) Pesticide Biochem. Physiol. 9:281-292.

1-55. (canceled)

56. A substantially purified and/or recombinant polypeptide comprising:
i) an amino acid sequence as provided in SEQ ID NO:1,
ii) an amino acid sequence which is at least 95% identical to i), and/or
iii) a biologically active fragment of i) or ii),
wherein the polypeptide is capable of hydrolysing a phenylurea, carbamate, and/or organophosphate.

57. The polypeptide of claim 56, which has one or more of the following features:
(i) the phenylurea is a N-dimethyl or N-methoxy-N-methyl substituted phenylurea,
(ii) the polypeptide has a lower Kₘ for diuron, chlortoluron, fluomethuron, metoxuron, and/or fenuron than a polypeptide comprising an amino acid sequence as provided in SEQ ID NO:3,
(iii) the carbamate is linuron ester or DMNPC,
(iv) the organophosphate is paraoxon ethyl,
(v) the polypeptide can be purified from a Mycobacterium sp, and
(vi) the polypeptide is fused to at least one other polypeptide.

58. An isolated and/or exogenous polynucleotide comprising:
i) a sequence of nucleotides as provided in SEQ ID NO:2,
ii) a sequence of nucleotides encoding the polypeptide according to claim 56, and/or
iii) a sequence of nucleotides which is at least 95% identical to i), and
wherein the polynucleotide encodes a polypeptide that hydrolyses a phenylurea, carbamate, and/or organophosphate.

59. A vector comprising at least one polynucleotide of claim 58.

60. A host cell comprising at least one polynucleotide of claim 58.

61. A method of producing a polypeptide comprising:
i) an amino acid sequence as provided in SEQ ID NO:1,
ii) an amino acid sequence which is at least 95% identical to i), and/or
iii) a biologically active fragment of i) or ii),
wherein the polypeptide is capable of hydrolysing a phenylurea, carbamate, and/or organophosphate,
the method comprising cultivating the host cell according to claim 60 under conditions which allow expression of the polynucleotide encoding the polypeptide, and recovering the expressed polypeptide.

62. A method of producing a polypeptide comprising:
i) an amino acid sequence as provided in SEQ ID NO:1,
ii) an amino acid sequence which is at least 95% identical to i), and/or
iii) a biologically active fragment of i) or ii),
wherein the polypeptide is capable of hydrolysing a phenylurea, carbamate, and/or organophosphate,
the method comprising cultivating the vector according to claim 59 under conditions which allow expression of the polynucleotide encoding the polypeptide, and recovering the expressed polypeptide.

63. An extract of the host cell of claim 60, wherein the extract comprises a polypeptide comprising:
i) an amino acid sequence as provided in SEQ ID NO:1,
ii) an amino acid sequence which is at least 83% identical to i), and/or
iii) a biologically active fragment of i) or ii),
wherein the polypeptide is capable of hydrolysing a phenylurea, carbamate, and/or organophosphate.

64. A composition for hydrolysing a phenylurea, carbamate, and/or organophosphate, the composition comprising the polypeptide according to claim 56.

65. A composition for hydrolysing a phenylurea, carbamate, and/or organophosphate, the composition comprising the extract of claim 63.

66. A method for hydrolysing a phenylurea, carbamate, and/or organophosphate, the method comprising contacting a phenylurea, carbamate, and/or organophosphate with the polypeptide according to claim 56.

67. A method for hydrolysing a phenylurea, carbamate, and/or organophosphate, the method comprising contacting a phenylurea, carbamate, and/or organophosphate with the extract according to claim 63.

68. A transgenic plant or non-human animal comprising an exogenous polynucleotide encoding at least one polypeptide according to claim 56.

69. A method of producing a polypeptide with enhanced ability to hydrolyse a phenylurea, carbamate, and/or organophosphate, or altered substrate specificity for a different type of phenylurea, carbamate, and/or organophosphate, the method comprising:
i) altering one or more amino acids of a first polypeptide according to claim 56,
ii) determining the ability of the altered polypeptide obtained from step i) to hydrolyse a phenylurea, carbamate, and/or organophosphate, and
iii) selecting an altered polypeptide with enhanced ability to hydrolyse a phenylurea, carbamate, and/or organophosphate, or altered substrate specificity for a different type of phenylurea, carbamate, and/or organophosphate, when compared to the polypeptide used in step i).
